(19) United States
(12) Patent Application Publication
(10) Pub. No.: US 2026/0038688 A1
(43) Pub. Date: Feb. 5, 2026

(54) METHODS AND SYSTEMS OF PLASMA CELL-FREE RNA AS NON-INVASIVE BIOMARKERS FOR PARKINSON'S DISEASE

(71) Applicants: Laura Ibanez, St. Louis, MO (US); Aleksandra Beric, St. Louis, MO (US)

(72) Inventors: Laura Ibanez, St. Louis, MO (US); Aleksandra Beric, St. Louis, MO (US)

(73) Assignee: Washington University, St. Louis, MO (US)

(21) Appl. No.: 19/288,820

(22) Filed: Aug. 1, 2025

Related U.S. Application Data
(60) Provisional application No. 63/678,680, filed on Aug. 2, 2024.

(51) Int. Cl. G16H 50/20; C12Q 1/6883; G16H 20/10

(52) U.S. Cl. CPC G16H 50/20; C12Q 1/6883; G16H 20/10; C12Q 2600/106; C12Q 2600/112

(57) ABSTRACT

Methods and systems for detecting, treating, and monitoring Parkinson's Disease (PD), as well as for differentiating PD from non-PD neurodegenerative diseases are provided. Methods include providing a biological sample from the subject; measuring a level of at least one plasma cell-free RNA (cfRNA) transcript in the biological sample; and determining a treatment for the subject based on the level of the at least one plasma cfRNA transcript. In some embodiments, the method further includes: determining a PD severity level based on the level of the at least one plasma cfRNA transcript; and determining the treatment for the subject further based on the PD severity level. Also provided are prediction models for identifying Parkinson's Disease in a subject in need thereof.

### CROSS-REFERENCE TO RELATED APPLICATIONS

This application claims the benefit of priority to U.S. Provisional Application Ser. No. 63/678,680 filed on 2 Aug. 2024, which is incorporated herein by reference in its entirety.

### STATEMENT OF FEDERALLY SPONSORED RESEARCH OR DEVELOPMENT

This invention was made with government support under AG062723 awarded by the National Institutes of Health and under W81XWH-20-1-0849 awarded by the Defense Health Agency, Medical Research and Development Branch. The government has certain rights in the invention.

### MATERIAL INCORPORATED BY REFERENCE

Applicants' publication entitled “Plasma acellular transcriptome contains Parkinson's disease signature that can inform clinical diagnosis” (medRxiv, October 2024) which includes all associated data in the form of supplementary material documents and tables, and which corresponds to the subject matter of the instant disclosure, is incorporated herein by reference in its entirety and as support for the present disclosure.

### FIELD

The present disclosure generally relates to differentiating Parkinson's Disease (PD) subjects from healthy controls, thereby enabling effective PD treatment.

### BACKGROUND

Parkinson's disease (PD) is a slowly progressing, complex neurodegenerative disorder, with higher prevalence in males. It is one of the most common neurodegenerative diseases (NDDs), second only to Alzheimer's disease (AD). As with other NDDs, the greatest risk factor for PD development is age, with incidence peaking after 80 years of age, with contributions from environmental and genetic factors. PD is characterized pathologically by formation of Lewy bodies (LBs) and early death of dopaminergic neurons, resulting in a typical clinical presentation including bradykinesia, rest tremor and rigidity. At the molecular level, LBs are primarily comprised of misfolded α-synuclein, which can spread between the cells, serving as a template for further α-synuclein misfolding.

While PD diagnoses largely depend on patient history and physical examination, no currently available tests enable definitive diagnosis of PD in the early stages. Instead, definitive diagnostics presently depends on neuropathological analyses upon death, typically occurring many years after disease onset. Several imaging methods can aid in confirm nigrostriatal deficits that occur in PD, but are not diagnostic. Dopamine transporter single-photon emission computed tomography (DaT SPECT) can detect cell loss in PD patients, while positron emission tomography (PET) scan can point to early signs of dopaminergic neuron damage. In addition to imaging, the field has strived to develop PD-specific cerebrospinal fluid (CSF) biomarkers, independent of clinical representation of the disease. CSF levels of α-synuclein have been the focal point of a number of studies. α-synuclein seed amplification assays (SAA) can differentiate between PD and healthy controls. However, results have varied, possibly due to clinical heterogeneity, cross-contamination with blood, or experimental differences, requiring further validation prior to clinical implementation. Lysosomal enzymes and neurofilament light chain emerged as candidates for biomarker panels, though they also require further investigation.

Another unmet need is the differential diagnosis of PD from other NDDs. Though it is a very common neurodegenerative disorder, PD is misdiagnosed in clinical practice with error rates reported to range from 15% to 24%. The prevailing reason for disagreement between clinical and neuropathological diagnoses is the heterogeneity of parkinsonism with non-PD pathologies, including dementia with Lewy bodies (DLB), multiple system atrophy (MSA) and progressive supranuclear palsy (PSP). Indeed, even established PD cases are greatly heterogeneous in the age of onset, rate of progression as well as clinical presentation, which led to the establishment of several PD subtypes.

Currently, there are no molecular diagnostic or prognostic biomarkers available for PD.

### BRIEF DESCRIPTION OF THE DISCLOSURE

Among the various aspects of the present disclosure is the provision of plasma cell-free RNA transcripts as non-invasive biomarkers for neurodegenerative disease.

In one aspect of the present disclosure, a method for treating Parkinson's Disease in a subject in need thereof is provided. The method comprises: providing a first biological sample obtained from the subject; measuring a first level of at least one plasma cell-free RNA (cfRNA) transcript in the first biological sample; and determining a first treatment for the subject based on the level of the at least one plasma cfRNA transcript.

In some embodiments, the method further comprises determining a PD severity level based on the level of the at least one plasma cfRNA transcript. In certain embodiments, determining the treatment for the subject is further based further on the determined PD severity level.

In some embodiments, the method further comprises determining a continuing treatment for the subject, wherein determining the continuing treatment comprises: providing a second biological sample obtained from the subject, wherein the second biological sample is obtained from the subject at a later time than the first biological sample is obtained from the subject; measuring a second level of the at least one plasma cfRNA transcript in the second biological sample; and determining a continuing treatment for the subject based on a change between the first and second levels of the at least one plasma cfRNA transcript.

In some embodiments, the at least one cfRNA transcript is a combination of at least 26 cfRNA transcripts. In certain embodiments, the combination of at least 26 cfRNA transcripts comprises FGR, SH3BP2, ATP5F1B, PTK2B, EMC3, PLAC8, TAF10, H2AC11, H2BC7, RERE, FCGR3A, and APOE. In other embodiments, the at least one cfRNA transcript is a combination of at least 87 cfRNA transcripts. In yet other embodiments, the at least one cfRNA transcript is a combination of at least 191 cfRNA transcripts.

In another aspect of the present disclosure, a method of differentiating Parkinson's Disease (PD) from a non-PD neurodegenerative disease in a subject in need thereof is provided. The method comprises: providing a biological sample from the subject; measuring a level of at least one plasma cell-free RNA (cfRNA) transcript in the biological sample; determining whether the subject has PD or a non-PD neurodegenerative disease based on the level of the at least one plasma cfRNA transcript.

In some embodiments, the subject is selected from a subject having, suspected of having, or at risk for developing PD and a subject having, suspected of having, or at risk for developing a non-PD neurodegenerative disease. In some embodiments, the non-PD neurodegenerative disease comprises Alzheimer's disease (AD), dementia with Lewy bodies (DLB), frontotemporal dementia (FTD), multiple system atrophy (MSA), progressive supranuclear palsy (PSP), Huntington's Disease (HD), and amyotrophic lateral sclerosis (ALS).

In some embodiments, the method further comprises, when the subject is determined to have PD, determining a PD severity level based on the level of the at least one plasma cfRNA transcript. In some embodiments, the method further comprises determining a treatment for the subject based further on the determined PD severity level.

In some embodiments, the at least one cfRNA transcript is a combination of at least 26 cfRNA transcripts. In certain embodiments, the combination of at least 26 cfRNA transcripts comprises FGR, SH3BP2, ATP5F1B, PTK2B, EMC3, PLAC8, TAF10, H2AC11, H2BC7, RERE, FCGR3A, and APOE. In other embodiments, the at least one cfRNA transcript is a combination of at least 87 cfRNA transcripts. In yet other embodiments, the at least one cfRNA transcript is a combination of at least 191 cfRNA transcripts.

In a further aspect of the present disclosure, a predictive model to identify Parkinson's Disease (PD) in a subject in need thereof is provided. The predictive model comprises: providing a level of at least one plasma cell-free RNA (cfRNA) transcript measured from a biological sample obtained from the subject; calculating a Kullback-Leibler divergence (KLD) value for each cfRNA transcript; ranking the KLD value for each cfRNA transcript; generating a L2 regularization linear model based on the ranked KLD values; computing an area under a curve (AUC) value for the L2 regularization linear model, wherein the curve is a receiver operating characteristic (ROC) curve; comparing the computed AUC value to a threshold value; and identifying PD in the subject if the AUC value meets the threshold value.

In some embodiments, the method further comprises, when PD is identified in the subject, determining a treatment for the subject based on the computed AUC value. In some embodiments, the threshold value is 0.85.

Other objects and features will be in part apparent and in part pointed out hereinafter.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings described herein are for illustrative purposes only. The drawings are not intended to limit the scope of the present teachings in any way.

FIG. 1 is a schematic showing the study design, including the study participants from two independent cohorts, the summary of the experimental approach (data processing and analysis), biological contextualization of the results via multiomic data integration with available independent datasets, and leveraging the results to build predictive models.

FIG. 2A is a schematic of multiomic integration between differential expression analysis of cfRNA (outer ring) with whole blood RNAseq (middle ring) and plasma proteomics (inner ring). The y-axes represent-log 10 of differential expression p-value, while x-axes represent genomic coordinates of the respective transcript/gene. Dotted lines symbolize the same transcript/gene or its protein product that are significantly differentially expressed across the multiple omic layers displayed.

FIG. 2B is a schematic of multiomic integration between differential expression analysis of cfRNA (outer ring) with brain RNAseq (middle ring) and CSF proteomics (inner ring). The y-axes represent-log 10 of differential expression p-value, while x-axes represent genomic coordinates of the respective transcript/gene. Dotted lines symbolize the same transcript/gene or its protein product that are significantly differentially expressed across the multiple omic layers displayed.

FIG. 2C is a graph showing single nuclei datasets for plasma cfRNA, substantia nigra, subcortical putamen, and cingulate cortex.

FIG. 2D is a schematic of multiomic integration between differential expression analysis of cfRNA (outer ring) with GWAS data (inner ring). The y-axes represent-log 10 of differential expression p-value, while x-axes represent genomic coordinates of the respective transcript/gene. Dotted lines symbolize the same transcript/gene or its protein product that are significantly differentially expressed across the multiple omic layers displayed.

FIG. 3A is a plot summarizing the significant result of KEGG pathway analysis. In orange are pathways first captured in all the predictive models and in blue are pathways captured in 87 and 191 transcript models.

FIG. 3B is a whisker plot showing the performance of the predictive models to differentiate between Parkinson's Disease (PD) and Huntington's disease (HD).

FIG. 3C is a whisker plot showing the performance of the predictive model to differentiate between Alzheimer's disease (AD), Dementia with Lewy bodies (DLB) and HC.

FIG. 3D is a whisker plot showing the performance of the predictive model to differentiate between AD, DLB, Frontotemporal dementia (FTD), and PD.

FIG. 4 is a volcano plot summarizing meta-analyses results. The Y-axis represents the −log 10 of the meta-analysis p-value, and the X-axis is the weighted mean of the log 2 fold change from the analysis of the individual populations. Significantly DE transcripts are highlighted in orange.

FIG. 5 is a graph showing model performance in the training population for the increasing KLD value cutoffs. The Y-axis represents the AUC of each model, X-axis represents the KLD value used as threshold, and in orange are highlighted the models selected for further analyses.

FIG. 6 is a heatmap comparing the log₂ fold change for the differential expression of the 214 transcripts included in the predictive models in each neurodegenerative disease. Transcripts are sorted in decreasing order of the log₂ fold change in the comparison between PD and healthy controls.

### DETAILED DESCRIPTION OF THE DISCLOSURE

Shown herein are methods to distinguish Parkinson's Disease (PD) from healthy controls and other neurodegenerative diseases using plasma cell-free RNA transcripts (cfRNAs). Using the cfRNAs, predictive models achieving high accuracy (area under the curve (AUC)=0.86) were generated in distinguishing PD from healthy controls. Plasma cfRNAs that associated with PD were identified and had high predictive value to differentiate PD from healthy controls. Leveraging two independent populations from two different movement disorder centers 2,188 differentially expressed cfRNAs were identified. The identified transcripts were enriched in PD relevant pathways, such as PD, ubiquitin-mediated proteolysis, and endocytosis. Utilizing transcriptomic and proteomic PD datasets, significant overlap with the results was found. Three predictive models were developed that distinguish PD from healthy control with an AUC≥0.85. Finally, it is shown herein that several of the predictive model transcripts significantly correlate with symptom severity. Overall, the present disclosure demonstrates the use of cfRNAs and predictive modeling to aid in PD diagnostics and monitoring.

The predictive models using cfRNA transcripts may comprise one or more of the following gene transcripts (Table 1). In one aspect, the predictive model may use a set of 10 or more transcripts. The predictive model may comprise a set of 26, 87, or 191 transcripts.

TABLE 1
PD cfRNA transcripts.
Gene Name

| | | | |
|---|---|---|---|
| ANK1 | EMC3 | NCOA2 | SEC31A |
| ANKRD9 | FAM110A | NDUFA4 | SEPTIN5 |
| ANP32B | FAUP1 | NECAP2 | SH3BP2 |
| ANXA5 | FCGR3A | NPM1P27 | SIAH2 |
| APOE | FGR | NUTF2 | SIPA1L3 |
| APP | FOXN3 | PABPC4 | SLC25A3 |
| APTK2B | GDF7 | PARP1 | SND1 |
| ARF4 | GGA2 | PARP14 | SNRNP200 |
| ARHGAP27 | GLUD1 | PBX1 | SNRPB |
| ARHGEF3 | GOLGA3 | PDK3 | SNRPN |
| ARL4C | GPRASP1 | PIK3AP1 | SNX20 |
| ARL8B | GTPBP2 | PLAC8 | SPIB |
| ATM | H2AC11 | PLCG2 | SRSF1 |
| ATP5F1B | H2BC11 | PLPBP | STT3B |
| ATP5FBP2 | H2BC7 | PNP | SYNE1 |
| BCL2 | H2BC9 | PRKD3 | SYNE2 |
| BIRC3 | H4C3 | PRKX | TAF10 |
| BMP6 | HCAK1 | PRRC2A | TAX1BP3 |
| BTG2 | HSPB1 | PSMD4 | THRAP3 |
| C10orf95-AS1 | IER2 | PSME3 | TMBIM1 |
| CA198 | IKZF3 | PTK2B | TMEM140 |
| CALD1 | IL32 | PYGL | TOMM20 |
| CARD8 | INPP5D | QKI | TPST2 |
| CCND2 | IQGAP1 | RAPGEF1 | TRIM33 |
| CDC42SE1 | ITCH | RASAL3 | TRIM44 |
| CDKN1C | ITGB1P1 | RASSF5 | TRIM58 |
| CDYL | JSRP1 | RBM3 | TSPYL1 |
| CLNS1A | KANSL1 | RBM8A | TTC7B |
| CLTC | LBH | RCSD1 | TUBA1A |
| CNDP2 | LGALS1 | RERE | TUBA1B-AS1 |
| CORO2B | LGALS8 | RHBDD1 | TULP4 |
| CPB2-AS1 | LINC01934 | RPH3A | TUT7 |
| CPEB4 | LST1 | RPL13A | UBA2 |
| CREBBP | MAFB | RPL13P12 | UBA52 |
| CTBP1 | MAN1A2 | RPL31 | UBE2G1 |
| CTCF | MAP3K1 | RPL34 | UBTF |
| DAD1 | MAP3K3 | RPL36A | USP10 |
| DHX15 | MBD2 | RPL37A | VPS37B |
| DNAJC27 | MBOAT2 | RPL41 | VSIR |
| DNAJC5 | MCTP1 | RPS11 | WARS1 |
| DOCK2 | MORC3 | RPS12 | XRCC5 |
| DOCK8 | MPIG6B | RPS27P8 | YWHAB |
| DTX3L | MTMR10 | RPS3AP6 | ZBTB7A |
| DUSP6 | MT-ND2 | RPS6 | ZC3H11A |
| EFCAB6 | MTR | RPS6KA1 | ZFP36L1 |
| EIF3H | MXD1 | RPSA | ZMAT2 |
| EIF3L | NACC2 | RUNX1 | ZNF217 |
| EIF4HP1 | NCF2 | SBF1 | |
| ELF4 | NCKAP1L | SCP2 | |

### Neurodegenerative Disease

The methods described herein can be used to detect, treat, and monitor a neurodegenerative disease, disorder, or condition.

For example, a neurodegenerative disease, disorder or condition can be a hereditary motor and sensory neuropathy (HMSN) (e.g., Charcot Marie Tooth (CMT) disease), CMT1 (a dominantly inherited, hypertrophic, predominantly demyelinating form), CMT2 (a dominantly inherited predominantly axonal form), Dejerine-Sottas (severe form with onset in infancy), CMTX (inherited in an X-linked manner), CMT4 (includes the various demyelinating autosomal recessive forms of Charcot-Marie-Tooth disease), hereditary sensory and autonomic neuropathy type IE, hereditary sensory and autonomic neuropathy type II, hereditary sensory and autonomic neuropathy type V, HMSN types 1A and 1B (e.g., dominantly inherited hypertrophic demyelinating neuropathies), HMSN type 2 (e.g., dominantly inherited neuronal neuropathies), HMSN type 3 (e.g., hypertrophic neuropathy of infancy [Dejerine-Sottas]), HMSN type 4 (e.g., hypertrophic neuropathy [Refsum] associated with phytanic acid excess), HMSN type 5 (associated with spastic paraplegia), or HMSN type 6 (e.g., with optic atrophy).

As another example, a neurodegenerative disease, disorder or condition can be Alzheimer's disease, amyotrophic lateral sclerosis (ALS), Alexander disease, Alpers' disease, Alpers-Huttenlocher syndrome, alpha-methylacyl-CoA racemase deficiency, Andermann syndrome, Arts syndrome, ataxia neuropathy spectrum, ataxia (e.g., with oculomotor apraxia, autosomal dominant cerebellar ataxia, deafness, and narcolepsy), autosomal recessive spastic ataxia of Charlevoix-Saguenay, Batten disease, beta-propeller protein-associated neurodegeneration, Cerebro-Oculo-Facio-Skeletal Syndrome (COFS), Corticobasal Degeneration, CLN1 disease, CLN10 disease, CLN2 disease, CLN3 disease, CLN4 disease, CLN6 disease, CLN7 disease, CLN8 disease, cognitive dysfunction, congenital insensitivity to pain with anhidrosis, dementia, familial encephalopathy with neuroserpin inclusion bodies, familial British dementia, familial Danish dementia, fatty acid hydroxylase-associated neurodegeneration, Gerstmann-Straussler-Scheinker Disease, GM2-gangliosidosis (e.g., AB variant), HMSN type 7 (e.g., with retinitis pigmentosa), Huntington's disease, infantile neuroaxonal dystrophy, infantile-onset ascending hereditary spastic paralysis, Huntington's disease (HD), infantile-onset spinocerebellar ataxia, juvenile primary lateral sclerosis, Kennedy's disease, Kuru, Leigh's Disease, Marinesco-Sjögren syndrome, Mild Cognitive Impairment (MCI), mitochondrial membrane protein-associated neurodegeneration, Motor neuron disease, Monomelic Amyotrophy, Motor neuron diseases (MND), Multiple System Atrophy, Multiple System Atrophy with Orthostatic Hypotension (Shy-Drager Syndrome), multiple sclerosis, multiple system atrophy, neurodegeneration in Down's syndrome (NDS), neurodegeneration of aging, Neurodegeneration with brain iron accumulation, neuromyelitis optica, pantothenate kinase-associated neurodegeneration, Opsoclonus Myoclonus, prion disease, Progressive Multifocal Leukoencephalopathy, Parkinson's disease (PD), PD-related disorders, polycystic lipomembranous osteodysplasia with sclerosing leukoencephalopathy, prion disease, progressive external ophthalmoplegia, riboflavin transporter deficiency neuronopathy, Sandhoff disease, Spinal muscular atrophy (SMA), Spinocerebellar ataxia (SCA), Striatonigral degeneration, Transmissible Spongiform Encephalopathies (Prion Diseases), or Wallerian-like degeneration.

### Modulation Agents

As described herein, gene and/or associated protein expression has been implicated in various diseases, disorders, and conditions. As such, modulation of gene and protein expression can be used for treatment of such conditions. A modulation agent can modulate response, such as by inducing or inhibiting gene and/or protein expression signaling. Modulation can comprise modulating protein expression on cells, modulating the quantity of gene/protein expressing cells, or modulating the quality of gene/protein expressing cells.

Modulation agents can be any composition or method that modulates expression on cells. For example, a modulation agent can be an activator, an inhibitor, an agonist, or an antagonist. As another example, the modulation can be the result of gene editing.

A modulation agent can be an antibody (e.g., a monoclonal antibody). A modulating agent can be an agent that induces or inhibits progenitor cell differentiation into gene/protein expressing cells.

Signal Reduction, Elimination, or Inhibition by Small Molecule Inhibitors, Shrna, siRNA, or Asos

As described herein, a modulation agent can be used for use in various therapies, such as to reduce/eliminate or enhance/increase expression signals. For example, a modulation agent can be a small molecule inhibitor, a short hairpin RNA (shRNA), or a short interfering RNA (siRNA). As another example, RNA (e.g., long noncoding RNA (lncRNA)) can be targeted with antisense oligonucleotides (ASOs) as a therapeutic. Processes for making ASOs targeted to RNAs are well known; see e.g., Zhou et al. 2016 Methods Mol Biol. 1402:199-213. Except as otherwise noted herein, therefore, the process of the present disclosure can be carried out in accordance with such processes.

### Inhibiting Agent

Inhibition of agents as described herein can be determined by standard pharmaceutical procedures in assays or cell cultures for determining the IC₅₀. The half maximal inhibitory concentration (IC₅₀) is a measure of the potency of a substance in inhibiting a specific biological or biochemical function. The IC₅₀ is a quantitative measure that indicates how much of a particular inhibitory substance (e.g., pharmaceutical agent or drug) is needed to inhibit, in vitro, a given biological process or biological component by 50%. The biological component could be an enzyme, cell, cell receptor, or microorganism, for example. IC₅₀ values are typically expressed as molar concentration. IC₅₀ is generally used as a measure of antagonist drug potency in pharmacological research. IC₅₀ is comparable to other measures of potency, such as EC₅₀ for excitatory drugs. EC₅₀ represents the dose or plasma concentration required for obtaining 50% of a maximum effect in vivo. IC₅₀ can be determined with functional assays or with competition binding assays.

### Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The term “transfection,” as used herein, refers to the process of introducing nucleic acids into cells by non-viral methods. The term “transduction,” as used herein, refers to the process whereby foreign DNA is introduced into another cell via a viral vector.

The terms “heterologous DNA sequence”, “exogenous DNA segment”, or “heterologous nucleic acid”, “transgene”, “exogenous polynucleotide” as used herein, each refers to a sequence that originates from a source foreign (e.g., non-native) to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Sequences described herein can also be the reverse, the complement, or the reverse complement of the nucleotide sequences described herein. The RNA goes in the reverse direction compared to the DNA, but its base pairs still match (e.g., G to C). The reverse complementary RNA for a positive strand DNA sequence will be identical to the corresponding negative strand DNA sequence. Reverse complement converts a DNA sequence into its reverse, complement, or reverse-complement counterpart.

| Base | Name | Bases Represented | Complementary Base |
|---|---|---|---|
| A | Adenine | A | T |
| T | Thymidine | T | A |
| U | Uridine(RNA only) | U | A |
| G | Guanidine | G | C |
| C | Cytidine | C | G |
| Y | pYrimidine | C T | R |
| R | puRine | A G | Y |
| S | Strong(3Hbonds) | G C | S* |
| W | Weak(2Hbonds) | A T | W* |
| K | Keto | T/U G | M |
| M | aMino | A C | K |
| B | not A | C G T | V |
| D | not C | A G T | H |
| H | not G | A C T | D |
| V | not T/U | A C G | B |
| N | Unknown | A C G T | N |

Complementarity is a property shared between two nucleic acid sequences (e.g., RNA, DNA), such that when they are aligned antiparallel to each other, the nucleotide bases at each position will be complementary. Two bases are complementary if they form Watson-Crick base pairs.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

An “expression vector”, otherwise known as an “expression construct”, is generally a plasmid or virus designed for gene expression in cells. The vector is used to introduce a specific gene into a target cell, and can commandeer the cell's mechanism for protein synthesis to produce the protein encoded by the gene. Expression vectors are the basic tools in biotechnology for the production of proteins. The vector is engineered to contain regulatory sequences that act as enhancer and/or promoter regions and lead to efficient transcription of the gene carried on the expression vector. The goal of a well-designed expression vector is the efficient production of protein, and this may be achieved by the production of significant amount of stable messenger RNA, which can then be translated into protein. The expression of a protein may be tightly controlled, and the protein is only produced in significant quantity when necessary through the use of an inducer, in some systems however the protein may be expressed constitutively. As described herein, Escherichia coli is used as the host for protein production, but other cell types may also be used.

In molecular biology, an “inducer” is a molecule that regulates gene expression. An inducer can function in two ways, such as:
(i) By disabling repressors. The gene is expressed because an inducer binds to the repressor. The binding of the inducer to the repressor prevents the repressor from binding to the operator. RNA polymerase can then begin to transcribe operon genes. An operon is a cluster of genes that are transcribed together to give a single messenger RNA (mRNA) molecule, which therefore encodes multiple proteins.
(ii) By binding to activators. Activators generally bind poorly to activator DNA sequences unless an inducer is present. An activator binds to an inducer and the complex binds to the activation sequence and activates target gene. Removing the inducer stops transcription. Because a small inducer molecule is required, the increased expression of the target gene is called induction.

Repressor proteins bind to the DNA strand and prevent RNA polymerase from being able to attach to the DNA and synthesize mRNA. Inducers bind to repressors, causing them to change shape and preventing them from binding to DNA. Therefore, they allow transcription, and thus gene expression, to take place.

For a gene to be expressed, its DNA sequence (or polynucleotide sequence) must be copied (in a process known as transcription) to make a smaller, mobile molecule called messenger RNA (mRNA), which carries the instructions for making a protein to the site where the protein is manufactured (in a process known as translation). Many different types of proteins can affect the level of gene expression by promoting or preventing transcription. In prokaryotes (such as bacteria), these proteins often act on a portion of DNA known as the operator at the beginning of the gene. The promoter is where RNA polymerase, the enzyme that copies the genetic sequence and synthesizes the mRNA, attaches to the DNA strand.

Some genes are modulated by activators, which have the opposite effect on gene expression as repressors. Inducers can also bind to activator proteins, allowing them to bind to the operator DNA where they promote RNA transcription. Ligands that bind to deactivate activator proteins are not, in the technical sense, classified as inducers, since they have the effect of preventing transcription.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “ribosome binding site”, or “ribosomal binding site (RBS)”, refers to a sequence of nucleotides upstream of the start codon of an mRNA transcript that is responsible for the recruitment of a ribosome during the initiation of translation. Generally, RBS refers to bacterial sequences, although internal ribosome entry sites (IRES) have been described in mRNAs of eukaryotic cells or viruses that infect eukaryotes. Ribosome recruitment in eukaryotes is generally mediated by the 5′ cap present on eukaryotic mRNAs.

A ribosomal skipping sequence (e.g., 2A sequence such as furin-GSG-T2A) can be used in a construct to prevent covalently linking translated amino acid sequences.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position+1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using self-replicating primers, paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above-required percent identities and retaining a required activity of the expressed protein is within the skill of the art. For example, directed evolution and rapid isolation of mutants can be according to methods described in references including, but not limited to, Link et al. (2007) Nature Reviews 5 (9), 680-688; Sanger et al. (1991) Gene 97 (1), 119-123; Ghadessy et al. (2001) Proc Natl Acad Sci USA 98 (8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A. For example, the percent identity can be at least 80% or about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

Substitution refers to the replacement of one amino acid with another amino acid in a protein or the replacement of one nucleotide with another in DNA or RNA. Insertion refers to the insertion of one or more amino acids in a protein or the insertion of one or more nucleotides with another in DNA or RNA. Deletion refers to the deletion of one or more amino acids in a protein or the deletion of one or more nucleotides with another in DNA or RNA. Generally, substitutions, insertions, or deletions can be made at any position so long as the required activity is retained.

“Point mutation” refers to when a single base pair is altered. A point mutation or substitution is a genetic mutation where a single nucleotide base is changed, inserted, or deleted from a DNA or RNA sequence of an organism's genome. Point mutations have a variety of effects on the downstream protein product-consequences that are moderately predictable based upon the specifics of the mutation. These consequences can range from no effect (e.g., synonymous mutations) to deleterious effects (e.g., frameshift mutations), with regard to protein production, composition, and function. Point mutations can have one of three effects. First, the base substitution can be a silent mutation where the altered codon corresponds to the same amino acid. Second, the base substitution can be a missense mutation where the altered codon corresponds to a different amino acid. Or third, the base substitution can be a nonsense mutation where the altered codon corresponds to a stop signal. Silent mutations result in a new codon (a triplet nucleotide sequence in RNA) that codes for the same amino acid as the wild type codon in that position. In some silent mutations the codon codes for a different amino acid that happens to have the same properties as the amino acid produced by the wild type codon. Missense mutations involve substitutions that result in functionally different amino acids; these can lead to alteration or loss of protein function. Nonsense mutations, which are a severe type of base substitution, result in a stop codon in a position where there was not one before, which causes the premature termination of protein synthesis and can result in a complete loss of function in the finished protein.

Generally, conservative substitutions can be made at any position so long as the required activity is retained. So-called conservative exchanges can be carried out in which the amino acid which is replaced has a similar property as the original amino acid, for example, the exchange of Glu by Asp, Gln by Asn, Val by 11e, Leu by 11e, and Ser by Thr. For example, amino acids with similar properties can be Aliphatic amino acids (e.g., Glycine, Alanine, Valine, Leucine, Isoleucine); hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6 (log 10 [Na⁺])+0.41 (fraction G/C content)-0.63 (% formamide)−(600/I). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transformed cells can be selected and propagated to provide recombinant host cells that comprise the expression vector stably integrated in the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| Side Chain Characteristic | Amino Acid |
|---|---|
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively | Charged |
| (Basic): | K R H |
| Negatively | Charged |
| (Acidic): | D E |

Conservative Substitutions III

| Original | Exemplary |
|---|---|
| Residue | Substitution |
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, |
| | Phe, |
| Leu (L) | Ile, Val, Met, Ala, |
| | Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| | Ile, Leu, Met, Phe, |
| Val (V) | Ala |

Exemplary nucleic acids that may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41 (1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), single guide RNA (sgRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14 (12): 807-15, describing targeting deoxyribonucleotide sequences; Lee et al. (2006) Curr Opin Chem Biol. 10, 1-8, describing aptamers; Reynolds et al. (2004) Nature Biotechnology 22 (3), 326-330, describing RNAi; Pushparaj and Melendez (2006) Clinical and Experimental Pharmacology and Physiology 33 (5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-ITT RNAi Designer, Invitrogen; siRNA Whitehead Institute Design Tools, Bioinformatics & Research Computing). Traits influential in defining optimal siRNA sequences include G/C content at the termini of the siRNAs, Tₘ of specific internal domains of the siRNA, siRNA length, position of the target sequence within the CDS (coding region), and nucleotide content of the 3′ overhangs.

### Genome Editing

As described herein, signals can be modulated (e.g., reduced, eliminated, or enhanced) using genome editing.

As described herein, activity, signals, expression, or function can be modulated (e.g., reduced, eliminated, or enhanced) using genome editing (e.g., upregulate, downregulate, overexpress, underexpress, express (e.g., transgenic expression), knock in, knock out, knockdown).

Processes for genome editing are well known; see e.g., Aldi 2018 Nature Communications 9 (1911). Except as otherwise noted herein, therefore, the process of the present disclosure can be carried out in accordance with such processes.

For example, genome editing can comprise CRISPR/Cas9, CRISPR-Cpf1, TALEN, or ZNFs. Adequate blockage by genome editing can result in protection from various diseases.

As an example, clustered regularly interspaced short palindromic repeats (CRISPR)/CRISPR-associated (Cas) systems are a new class of genome-editing tools that target desired genomic sites in mammalian cells. Recently published type II CRISPR/Cas systems use Cas9 nuclease that is targeted to a genomic site by complexing with a synthetic guide RNA that hybridizes to a 20-nucleotide DNA sequence and immediately preceding an NGG motif recognized by Cas9 (thus, a (N) 20NGG target DNA sequence). This results in a double-strand break three nucleotides upstream of the NGG motif. The double strand break instigates either non-homologous end-joining, which is error-prone and conducive to frameshift mutations that knock out gene alleles, or homology-directed repair, which can be exploited with the use of an exogenously introduced double-strand or single-strand DNA repair template to knock in or correct a mutation in the genome. Thus, genomic editing, for example, using CRISPR/Cas systems could be useful tools for therapeutic applications to target cells by the removal or addition of signals (e.g., activate (e.g., CRISPRa), upregulate, overexpress, downregulate).

For example, the methods as described herein can comprise a method for altering a target polynucleotide sequence in a cell comprising contacting the polynucleotide sequence with a clustered regularly interspaced short palindromic repeats-associated (Cas) protein.

### Gene Therapy and Genome Editing

Gene therapies can include inserting a functional gene with a viral vector. Gene therapies are rapidly advancing.

There has recently been an improved landscape for gene therapies. For example, in the first quarter of 2019, there were 372 ongoing gene therapy clinical trials (Alliance for Regenerative Medicine, May 9, 2019).

Any vector known in the art can be used. For example, the vector can be a viral vector selected from retrovirus, lentivirus, herpes, adenovirus, adeno-associated virus (AAV), rabies, Ebola, lentivirus, or hybrids thereof.

Gene therapy strategies.

| | Strategy |
|---|---|
| Viral Vectors | |
| Retroviruses | Retroviruses are RNA viruses transcribing |
| | their single-stranded |
| | genome into a double-stranded DNA copy, |
| | which can integrate into host chromosome |
| Adenoviruses (Ad) | Ad can transfect a variety of quiescent and |
| | proliferating |
| | cell types from various species and can |
| | mediate |
| | robust gene expression |
| Adeno-associated | Recombinant AAV vectors contain no viral |
| Viruses (AAV) | DNA and can carry ~4.7 kb of foreign |
| | transgenic material. They |
| | are replication defective and can replicate |
| | only while |
| | coinfecting with a helper virus |
| Non-viral vectors | |
| plasmid DNA | pDNA has many desired characteristics as a |
| (pDNA) | gene |
| | therapy vector; there are no limits on the size |
| | or genetic |
| | constitution of DNA, it is relatively |
| | inexpensive to supply, |
| | and unlike viruses, antibodies are not |
| | generated |
| | against DNA in normal individuals |
| RNAi | RNAi is a powerful tool for gene specific |
| | silencing that |
| | could be useful as an enzyme reduction |
| | therapy or |
| | means to promote read-through of a |
| | premature stop |
| | codon |

Gene therapy can allow for the constant delivery of the enzyme directly to target organs and eliminates the need for weekly infusions. Also, correction of a few cells could lead to the enzyme being secreted into the circulation and taken up by their neighboring cells (cross-correction), resulting in widespread correction of the biochemical defects. As such, the number of cells that must be modified with a gene transfer vector is relatively low.

Genetic modification can be performed either ex vivo or in vivo. The ex vivo strategy is based on the modification of cells in culture and transplantation of the modified cell into a patient. Cells that are most commonly considered therapeutic targets for monogenic diseases are stem cells. Advances in the collection and isolation of these cells from a variety of sources have promoted autologous gene therapy as a viable option.

The use of endonucleases for targeted genome editing can solve the limitations presented by the usual gene therapy protocols. These enzymes are custom molecular scissors, allowing cutting DNA into well-defined, perfectly specified pieces, in virtually all cell types. Moreover, they can be delivered to the cells by plasmids that transiently express the nucleases, or by transcribed RNA, avoiding the use of viruses.

### Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc., may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently, affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

### Therapeutic Methods

Also provided is a process of treating, preventing, or reversing Parkinson's Disease (PD) in a subject in need thereof via accurate diagnosis based on one or more non-invasive plasma cfRNA biomarkers, so as to determine and administer an effective PD treatment.

Methods described herein are generally performed on a subject in need thereof. A subject in need of the therapeutic methods described herein can be a subject having, diagnosed with, suspected of having, or at risk for developing Parkinson's Disease. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of a therapeutic agent is, for example, an amount that would cause the desired therapeutic effect in a subject while minimizing undesired side effects. In various embodiments, an effective amount of a therapeutic agent described herein can substantially inhibit, slow the progress of, or limit the development of PD.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of a therapeutic agent can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to effectively treat PD based at least in part on one or more plasma cfRNA non-invasive biomarker levels.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; the activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes reversing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or a physician.

Administration of a therapeutic agent can occur as a single event or over a time course of treatment. For example, a therapeutic agent can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to or before, concurrent with, or after conventional treatment modalities for PD.

A therapeutic agent can be administered simultaneously or sequentially with another agent, such as an antibiotic, an anti-inflammatory, or another agent. For example, a therapeutic agent can be administered simultaneously with another agent, such as an antibiotic or an anti-inflammatory. Simultaneous administration can occur through administration of separate compositions, each containing one or more of a therapeutic agent, an antibiotic, an anti-inflammatory, or another agent. Simultaneous administration can occur through administration of one composition containing two or more of a therapeutic agent, an antibiotic, an anti-inflammatory, or another agent. A therapeutic agent can be administered sequentially with an antibiotic, an anti-inflammatory, or another agent. For example, a therapeutic agent can be administered before or after administration of an antibiotic, an anti-inflammatory, or another agent.

Active compounds are administered at a therapeutically effective dosage sufficient to treat a condition associated with a condition in a patient. For example, the efficacy of a compound can be evaluated in an animal model system that may be predictive of efficacy in treating the disease in a human or another animal, such as the model systems shown in the examples and drawings.

An effective dose range of a therapeutic can be extrapolated from effective doses determined in animal studies for a variety of different animals. In general, a human equivalent dose (HED) in mg/kg can be calculated in accordance with the following formula (see e.g., Reagan-Shaw et al., FASEB J., 22 (3): 659-661, 2008, which is incorporated herein by reference):

$$HED ( mg / kg ) = Animal dose ( mg / kg ) \times ( Animal {K}_{m} / Human {K}_{m} )$$

Use of the Kₘ factors in conversion results in more accurate HED values, which are based on body surface area (BSA) rather than only on body mass. Kₘ values for humans and various animals are well known. For example, the Kₘ for an average 60 kg human (with a BSA of 1.6 m2) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are peculiar to each individual. Nonetheless, a calculated HED dose provides a general guide. Other factors affecting the dose include the physical and clinical state of the patient, the route of administration, the intended goal of treatment, and the potency, stability, and toxicity of the particular therapeutic formulation.

The actual dosage amount of a compound of the present disclosure or composition comprising a compound of the present disclosure administered to a subject may be determined by physical and physiological factors such as type of animal treated, age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

In some embodiments, the therapeutic agent may be administered in an amount from about 1 mg/kg to about 100 mg/kg, or about 1 mg/kg to about 50 mg/kg, or about 1 mg/kg to about 25 mg/kg, or about 1 mg/kg to about 15 mg/kg, or about 1 mg/kg to about 10 mg/kg, or about 1 mg/kg to about 5 mg/kg, or about 3 mg/kg. In some embodiments, a therapeutic agent may be administered in a range of about 1 mg/kg to about 200 mg/kg, or about 50 mg/kg to about 200 mg/kg, or about 50 mg/kg to about 100 mg/kg, or about 75 mg/kg to about 100 mg/kg, or about 100 mg/kg.

The effective amount may be less than 1 mg/kg/day, less than 500 mg/kg/day, less than 250 mg/kg/day, less than 100 mg/kg/day, less than 50 mg/kg/day, less than 25 mg/kg/day or less than 10 mg/kg/day. It may alternatively be in the range of 1 mg/kg/day to 200 mg/kg/day.

In other non-limiting examples, a dose may also comprise from about 1 micro-gram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

### Cell Therapy

Cells generated according to the methods described herein can be used in cell therapy. Cell therapy (also called cellular therapy, cell transplantation, or cytotherapy) can be a therapy in which viable cells are injected, grafted, or implanted into a patient in order to effectuate a medicinal effect or therapeutic benefit. For example, transplanting T-cells capable of fighting cancer cells via cell-mediated immunity can be used in the course of immunotherapy, grafting stem cells can be used to regenerate diseased tissues, or transplanting beta cells can be used to treat diabetes.

Stem cell and cell transplantation has gained significant interest by researchers as a potential new therapeutic strategy for a wide range of diseases, in particular for degenerative and immunogenic pathologies.

Allogeneic cell therapy or allogenic transplantation uses donor cells from a different subject than the recipient of the cells. A benefit of an allogeneic strategy is that unmatched allogenic cell therapies can form the basis of “off the shelf” products.

Autologous cell therapy or autologous transplantation uses cells that are derived from the subject's own tissues. It could also involve the isolation of matured cells from diseased tissues, to be later re-implanted at the same or neighboring tissues. A benefit of an autologous strategy is that there is limited concern for immunogenic responses or transplant rejection.

Xenogeneic cell therapies or xenotransplantation uses cells from another species. For example, pig derived cells can be transplanted into humans. Xenogeneic cell therapies can involve human cell transplantation into experimental animal models for assessment of efficacy and safety or enable xenogeneic strategies to humans as well.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency; improve taste of the product; or improve shelf life of the product.

### Screening

Also provided are screening methods.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 MW, or less than about 1000 MW, or less than about 800 MW) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules; and electronic libraries of commercial compounds provided by vendors, for example, ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character xlogP of about-2 to about 4). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character xlogP of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict the bioavailability of a compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

### Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to systems, models, and algorithms disclosed herein. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or another substrate, and/or may be supplied as an electronic-readable medium or video. Detailed instructions may not be physically associated with the kit; instead, a user may be directed to an Internet web site specified by the manufacturer or distributor of the kit.

A control sample or a reference sample as described herein can be a sample from a healthy subject or sample, a wild-type subject or sample, or from populations thereof. A reference value can be used in place of a control or reference sample, which was previously obtained from a healthy subject or a group of healthy subjects or a wild-type subject or sample. A control sample or a reference sample can also be a sample with a known amount of a detectable compound or a spiked sample.

The methods and algorithms of the invention may be enclosed in a controller or processor. Furthermore, methods and algorithms of the present invention, can be embodied as a computer-implemented method or methods for performing such computer-implemented method or methods, and can also be embodied in the form of a tangible or non-transitory computer-readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer program include, for example, floppy disks and diskettes, compact disk (CD)-ROMs (whether or not writeable), DVD digital disks, RAM and ROM memories, computer hard drives and back-up drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general-purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41 (1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Plasma Acellular Transcriptome Contains Parkinson'S Disease Signature that can Inform Clinical Diagnosis

This example identifies plasma cell-free transcripts (cfRNA) associated with Parkinson's disease (PD) that can also be leveraged to differentiate PD from healthy controls in a minimally invasive manner. Leveraging two independent populations from two movement disorder centers 2,188 differentially expressed cfRNAs were identified after meta-analysis. The identified transcripts were enriched in PD relevant pathways, such as PD (p=9.26×10⁻⁴), ubiquitin-mediated proteolysis (p=7.41×10⁻⁵) and endocytosis (p=4.21×10⁻⁶). Utilizing brain, whole blood, and acellular plasma transcriptomic and proteomic PD datasets, significant overlap was found across dysregulated biological species in the different tissues and biological layers. Three predictive models were developed containing increasing number of transcripts that can distinguish PD from healthy control with an area under the ROC Curve (AUC) ≥0.85. Finally, several of the predictive transcripts were shown to significantly correlate with symptom severity measured by UPDRS-III. Overall, the present disclosure has demonstrated that cfRNA contains pathological signatures and has the potential to be utilized as biomarker to aid in PD diagnostics and monitoring.

### INTRODUCTION

Parkinson's disease (PD) is a slowly progressing, complex neurodegenerative disorder, with higher prevalence in males. It is one of the most common neurodegenerative diseases (NDDs), second only to Alzheimer's disease (AD). As with other NDDs, the greatest risk factor for PD development is age, with incidence peaking after 80 years of age, with contributions from environmental and genetic factors. PD is characterized pathologically by formation of Lewy bodies (LBs) and early death of dopaminergic neurons, resulting in a typical clinical presentation including bradykinesia, rest tremor and rigidity. Other clinical hallmarks of PD include a number of non-motor symptoms, such as sleep, gastrointestinal and olfactory disorders, which may precede motor disorders by over a decade. At the molecular level, LBs are primarily comprised of misfolded α-synuclein, which can spread between the cells, serving as a template for further α-synuclein misfolding.

While PD diagnoses largely depend on patient history and physical examination, no currently available tests enable definitive diagnosis of PD in the early stages. Instead, definitive diagnostics presently depends on neuropathological analyses upon death, typically occurring many years after disease onset. Several imaging methods can aid in confirming nigrostriatal deficits that occur in PD but are not diagnostic. Dopamine transporter single-photon emission computed tomography (DaT SPECT) can detect cell loss in PD patients, while positron emission tomography (PET) scan can point to early signs of dopaminergic neuron damage. Conversely, magnetic resonance imaging (MRI) methods provide modest benefit for diagnosis of PD. In addition to imaging, the field has strived to develop PD-specific cerebrospinal fluid (CSF) biomarkers, independent of clinical representation of the disease. CSF levels of α-synuclein have been the focal point of a number of studies. α-synuclein seed amplification assays (SAA) have shown a lot of promise, with the ability to differentiate between PD and healthy controls. However, results have been variable possibly due to clinical heterogeneity, cross-contamination with blood, or experimental differences, requiring further validation prior to clinical implementation. Lysosomal enzymes and neurofilament light chain emerged as candidates for biomarker panels, though they also require further investigation. Another unmet need is the differential diagnosis of PD from other NDDs. Though it is a very common neurodegenerative disorder, PD is misdiagnosed in clinical practice with error rates reported to range from 15% to 24%. The prevailing reason for disagreement between clinical and neuropathological diagnoses is the heterogeneity of parkinsonism with non-PD pathologies, including dementia with Lewy bodies (DLB), multiple system atrophy (MSA) and progressive supranuclear palsy (PSP). Indeed, even established PD cases are greatly heterogeneous in the age of onset, rate of progression as well as clinical presentation, which led to the establishment of several PD subtypes.

While most blood-based biomarker studies focus on measuring levels of various proteins, circulating nucleic acids have found their place in clinical practice. Analyses of cell-free DNA (cfDNA) have revolutionized the field of obstetrics and antenatal testing by allowing the identification of fetal aneuploidies in a sample of mother's blood, thus reducing test-related risk of miscarriage. cfDNA has been utilized as a biomarker for cancer, metabolic disorders, and a way to assess the health of donor organs in a recipient's body upon organ transplantation. In addition to cfDNA, cell-free RNA (cfRNA) can also be captured from plasma and provides a temporal snapshot of cellular processes throughout the body, as it is released from cells as part of normal cell death. Numerous studies are investigating the potential of using cfRNAs as biomarkers for prenatal testing, cancer, and AD. Furthermore, a recent study provided evidence of circulating micro RNAs being involved in the regulation of PD-associated genes, adding support to the utility of non-protein biomarkers in the PD field.

As described herein, plasma cfRNA from two independent populations of PD participants was used to capture transcriptional changes caused by PD pathology. The findings were biologically contextualized via pathway analyses and multiomic data integration by accessing whole blood and brain transcriptomic datasets and plasma and CSF proteomic datasets. They were then leveraged to build a predictive model that could accurately predict PD using a limited number of transcripts, with the potential for translation into clinical practice. The capabilities of the best performing models were further evaluated to discriminate between PD and AD, DLB and frontotemporal dementia (FTD) to ensure that captured changes were specific to PD pathobiology.

### Methods

### Study Design

Acellular RNAseq data was analyzed from two independent movement disorder clinical cohorts, Hospital Universitari Mutua Terrassa (HUMT) in Barcelona, Spain, and Washington University in Saint Louis School of Medicine (WashU) in Saint Louis, US. Differential expression (DE) analyses were performed in HUMT (206 participants) and WashU (175 participants) cohorts separately, followed by meta-analysis. Transcripts with Benjamini-Hochberg corrected p-values below 0.05 in the meta-analysis were considered DE. To understand the biological significance of the DE transcripts, pathway analyses was performed and in-house and publicly available datasets were leveraged to contextualize the expression of the identified transcripts in whole blood and brain, as well as the accumulation of corresponding proteins in plasma and CSF. To assess the diagnostic capabilities of cfRNAs, several predictive models were developed, focusing on the DE transcripts. A third independent dataset was then employed, consisting of participants with dementia with DLB, AD, and FTD, to test the specificity of the predictive models for PD. AD, DLB, and FTD participants were diagnosed according to the clinical criteria contained in the Uniform Data Set (UDS), the standard set of clinical data collected in all participants enrolled in any of the 37-federally funded ADRCs. Finally, utilizing the information available about participants' motor symptom severity, measured by Unified Parkinson's Disease Rating Scale Part III (UPDRS-III), and cognitive status measured by The Montreal Cognitive Assessment (MoCA), the clinical relevance of transcripts included in the predictive models was assessed.

### Study Participants

Plasma samples were obtained from two independent cohorts, HUMT and WashU. HUMT cohort included a total of 206 plasma samples (87 PD participants and 119 healthy controls), while the WashU cohort included 175 samples (94 PD participants and 81 healthy controls). All samples available from the two cohorts were included to maximize the power of the analyses. Given the different geographical location and standards of care, the two cohorts show some differences (Table 2). Both populations are composed exclusively of participants of European ancestry. They are comparable in proportion of female participants (HUMT 47.57%, WashU 48.57%; p=0.93). The HUMT population shows a lower mean age (68.26±8.39) compared to the WashU population (72.97±6.78; p=3.30×10⁻⁹). Differences are also observed in motor symptom severity as measured by the UPDRS-III scale. Participants in the WashU population displayed greater UPDRS-III (26.03±9.10), compared to HUMT participants (18.69±8.99; p=6.39×10⁻⁴). Finally, dementia was assessed only in the WashU participants, who presented with mild or no signs of dementia (25.32±4.22), as measured by the MoCA scale. Similarly, no therapeutic information was available for the HUMT dataset.

### RNA Extraction and Sequencing

Whole blood samples were collected from all participants. Within 20 min of collection, blood samples were centrifuged for 10 min at 1500 rpm to obtain plasma and subsequently stored at −80° C. Plasma samples were thawed on ice and centrifuged for 5 min at 2000 rpm prior to RNA extraction to remove any cells present and avoid cellular RNA contamination. Total plasma cfRNA was extracted from 0.5 mL of plasma using the Maxwell RSC miRNA from plasma or serum kit (Ambion) and ribodepleted (NEBNext rRNA Depletion Kit). After total RNA quantification, libraries were generated using the NEBNext Ultra II Directional RNA Library Prep Kit for Illumina (New England Biolabs) using 1 ng of RNA as input. Libraries were cleaned for adapter dimers prior to sequencing. 40 million 100 base pair single-end reads were targeted for each sample using an Illumina NovaSeq 6000.

### Data Processing and Quality Control

FastQC (v0.11.7) was used to evaluate the sequencing quality of each sample. Reads were aligned to the human reference genome GRCh38 using STAR (v2.7.1a). The quality of sequences and alignments was assessed with PICARD (v2.26) and SAMtools, and transcripts quantified using Salmon (v0.11.3). Quality control measures were gathered via MultiQC (v1.9) followed by stringent quality control (QC). Briefly, all genes with less than ten reads in 90% or more of the individuals were removed. Subsequently, transcriptome Principal Component Analysis (PCA) were performed and screened for correlation with technical and methodological variables to detect potential biases. A strong correlation was observed with total reads and coding bases; thus, all samples with less than 10% of coding bases and less than 1,000,000 total reads were removed. Outlier samples identified via transcriptome PCA, defined as samples whose first two principal component values deviated more than three standard deviations from mean values of either of the respective principal components, were also removed.

Despite following the same protocol for data generation, processing, and QC, the two datasets included in the present study (HUMT and WashU) were sequenced at different timepoints. Each dataset underwent QC separately. ComBat_seq function from the sva package was used to adjust for technical variation within each dataset. Batch effect correction was followed by PCA and removal of any additional outliers. There is RNA degradation associated with plasma long-term storage (up to 20 years), consequently, degradation was addressed using DESeq2 (v1.22.2) to find transcripts associated with storage time in control participants as previously reported. All transcripts nominally (p<0.05) associated with storage time were removed from the analyses from both HUMT (n=486 transcripts) and WashU (n=221 transcripts) datasets. Further, due to prevalence of PD therapies, its effect on the transcriptome, and lack of medication data for HUMT population, transcripts associated with PD-medication usage when comparing PD treated participants and PD not treated were identified in the WashU dataset using DESeq2. Any nominally significant transcripts (p<0.05) were removed from further analyses from both datasets (n=630 transcripts) to minimize spurious associations due to medication. Overall, 27,832 transcripts passed QC and were included used in subsequent analyses.

### Differential Expression Analyses and Pathway Analyses

Differential expression (DE) analyses were performed using DESeq2 in HUMT and WashU data separately, followed by meta-analysis using metaRNASeq. All analyses were adjusted by sex and age at blood draw. Benjamini-Hochberg correction (FDR) was used to correct for multiple testing, considering meta-analysis FDR p-values lower than 0.05 as significant. No effect size (log 2 fold change) value cut-off was applied. Pathway enrichment analyses were carried out using clusterProfiler to functionally characterize the identified transcripts and FDR p-values below 0.05 were regarded as significant.

### Multi-Omic Data Integration

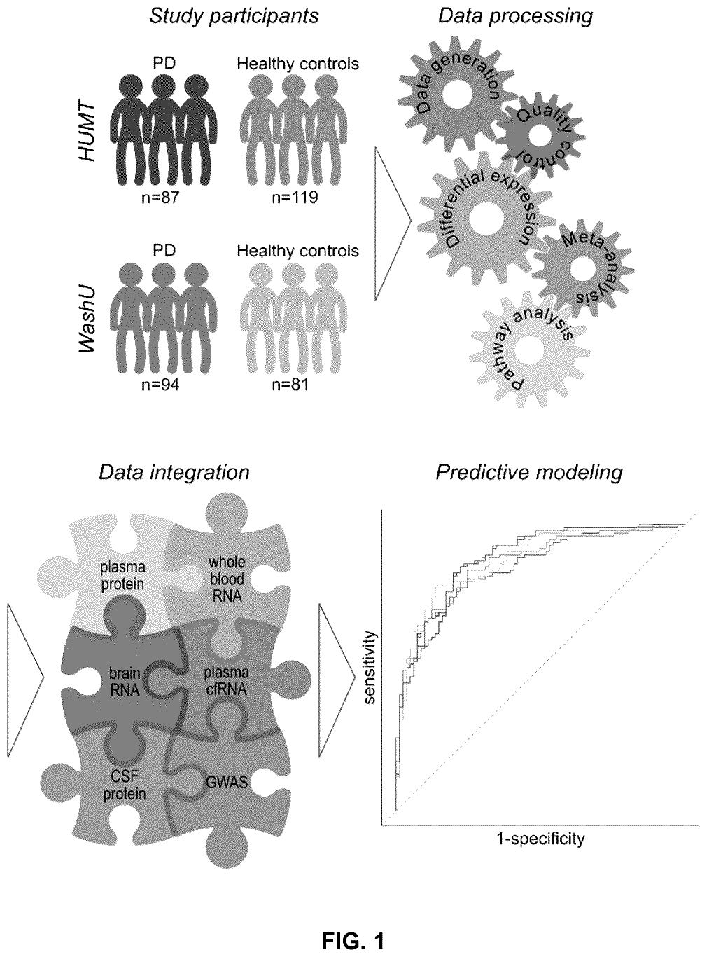

To biologically contextualize the findings, the results from the cfRNA DE meta-analysis were compared with: (i) in-house brain bulk RNAseq, (ii) publicly available, whole blood bulk RNAseq, (iii) publicly available plasma and (iv) CSF proteomic data, and (v) in house and (vi) publicly available brain single-cell RNAseq data including substantia nigra scRNAseq (FIG. 1). Following differential expression or accumulation analyses in each of the RNAseq or proteomic datasets, the overlap was identified between nominally significant findings and the DE plasma acellular transcripts and each accessed omic dataset. The significance of each overlap was tested using hypergeometric test (phyper function in R), p-values lower than 0.05 considered significant.

Additionally, to investigate whether any of the DE transcripts mapped to PD associated loci identified by genome wide association analyses (GWAS), the information available in the PD GWAS locus browser was leveraged and in the latest multi-ancestry genome-wide association study. With the PD GWAS locus browser, the scores were recorded that rank the genes based on the amount of supporting evidence compiled in the PD GWAS locus browser. Next, to compare the results to the multi-ancestry GWAS, locations were converted of all lead SNPs from hg19 version of the human genome to the respective locations in hg38 version of the human genome and then found coordinates 500 kb upstream and downstream from each SNP. All regions that overlapped after the coordinate conversion were collapsed into 69 non-overlapping genomic regions. Subsequently, collapsed PD-associated genomic regions were overlapped with genomic start and end coordinates of the identified DE genes using bedtools intersect from BEDTools tool suite.

### Predictive Models Construction and Evaluation

In brief, to build and evaluate predictive models, glmnet (v4.1.6) was leveraged to produce a suitable classifier to identify PD cases based on plasma acellular gene expression. HUMT was used as the training and WashU as the testing dataset. After ComBat_seq regression, transcript counts were further scaled between the two datasets by computing the z-score using the mean and standard deviation. With the transcripts that were significantly DE in the meta-analysis after multiple test correction (FDR<0.05), Kullback-Leibler divergence (KLD) was calculated between the training (HUMT) and the testing (WashU) dataset for each transcript using R package entropy v1.3.1. A hundred L2 regularization (lasso regression) linear models were trained with increasing number of transcripts, ranging in KLD value from 0.01 to 1 in increments of 0.01. The Area Under the Receiver Operating Characteristic (ROC) Curve (AUC) value was computed for all models in the training dataset.

### Specificity Analyses

Performance of the best predictive models was evaluated in 44 AD, 16 FTD and 17 DLB cases from a publicly available dataset from the Knight-ADRC, as well as twelve DLB participants available in the HUMT dataset. Knight-ADRC data has been generated and processed as described above for the HUMT and WashU datasets. Transcript counts were scaled by computing the z-score using the mean and standard deviation. Then, risk score for each individual was calculated using the previously defined predictive models. Scores higher than 0.50 were considered cases. ROC curve was computed by comparing the predicted to the actual disease status for each compared group. The ability of the predictive models was assessed to differentiate between AD, DLB or FTD and healthy controls, as well as between AD, DLB or FTD and PD. Additionally, the addition of the APOE genotype to the cfRNA predictor was evaluated for improving the model performance in differentiating between PD and AD, as APOE is a crucial genetic risk factor for AD. APOE genotype data was available for AD and WashU PD participants. To discern whether the effect of APOE was captured by the predictor, the APOE genotype was included in the model coded by two variables representing the number of ε2 alleles and ε4 alleles.

### Evaluation of Predictive Models' Clinical Relevance

To explore the relationship of selected transcripts with PD clinical manifestations, Spearman correlations were calculated between normalized and age and sex adjusted transcript counts with UPDRS-III or MoCA scores for those PD participants with available data. UPDRS-III information was available for 76 participants from HUMT and 27 participants in the WashU population. To maximize sample size and statistical power for this exploratory analysis, the two populations were combined for correlation with UPDRS-III. MoCA scores were not available for the HUMT population, thus correlation to MoCA analysis was carried out only in the WashU population (n=28). Expression levels for each transcript were ranked and rank was used to calculate correlations to UPDRS-III or MoCA via Spearman correlation analyses. Correlations were considered significant if p-value was lower than 0.05.

### Results

### Acellular Transcriptomic Patterns Translate to Changes of Plasma Protein Abundances.

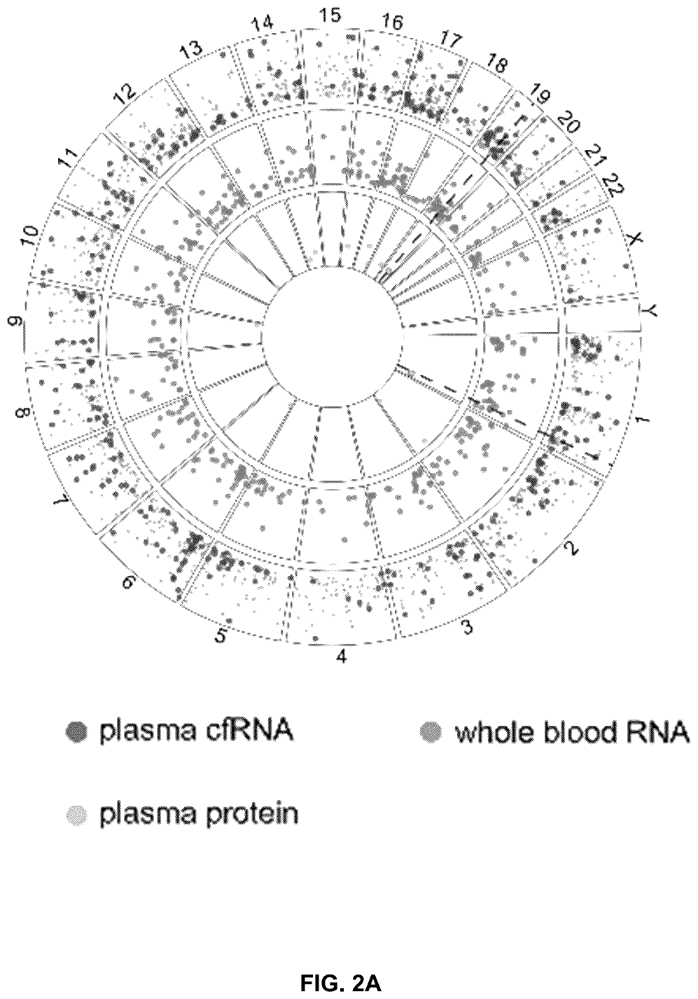

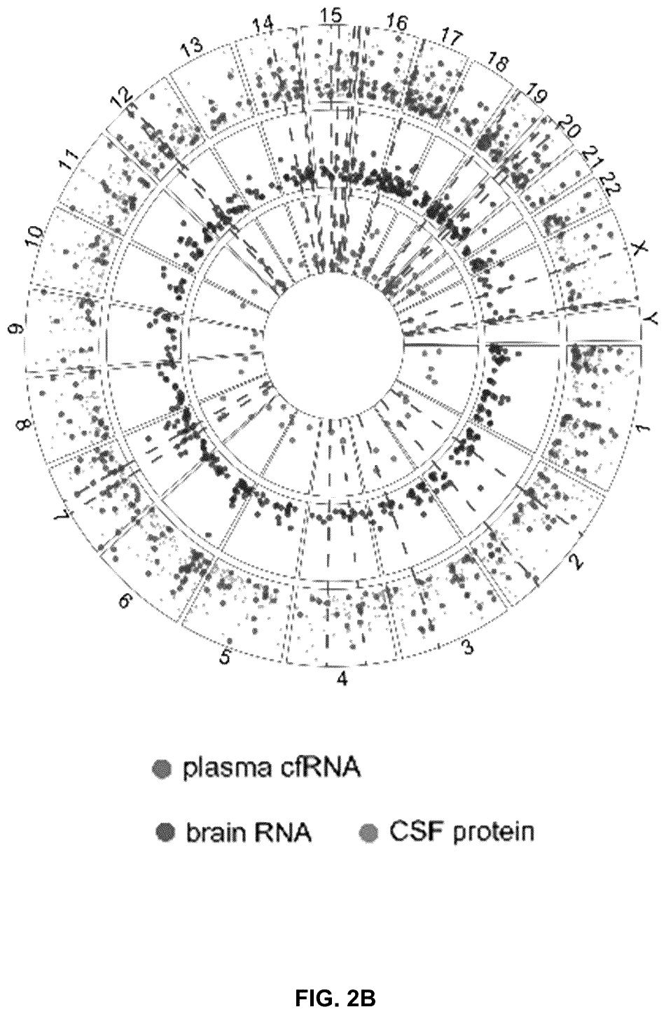

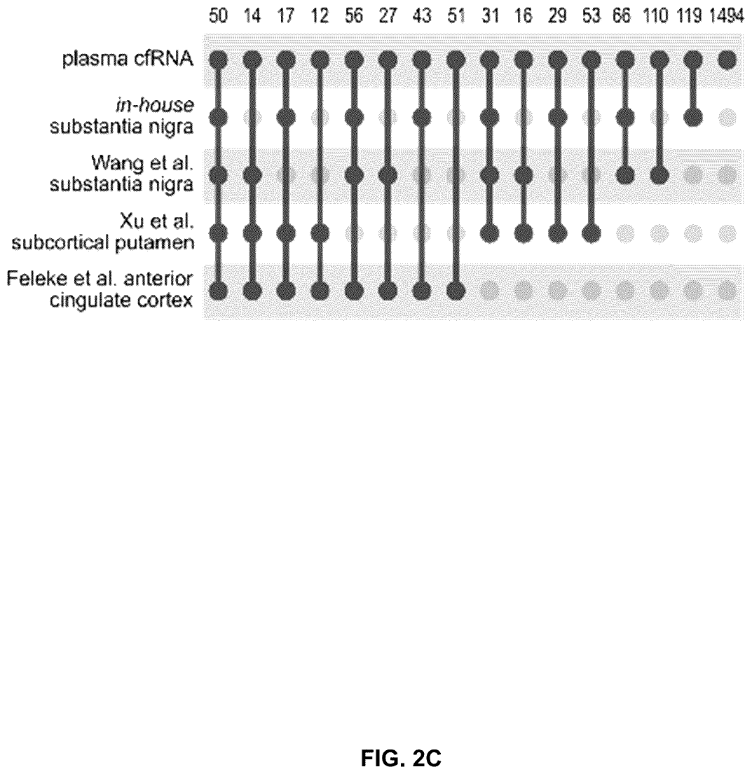

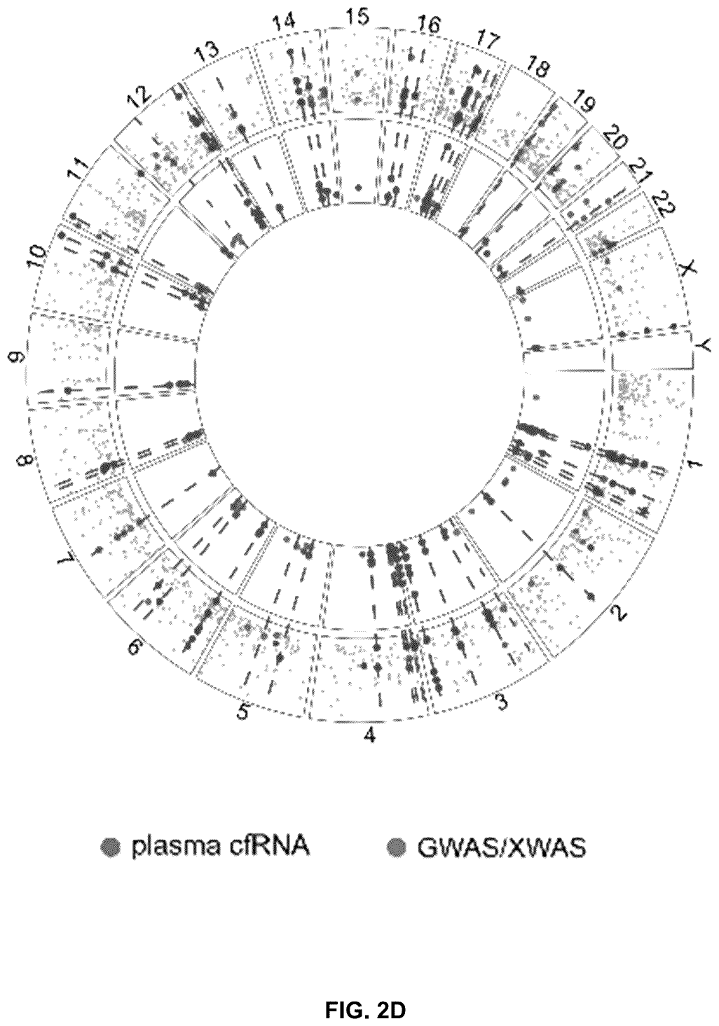

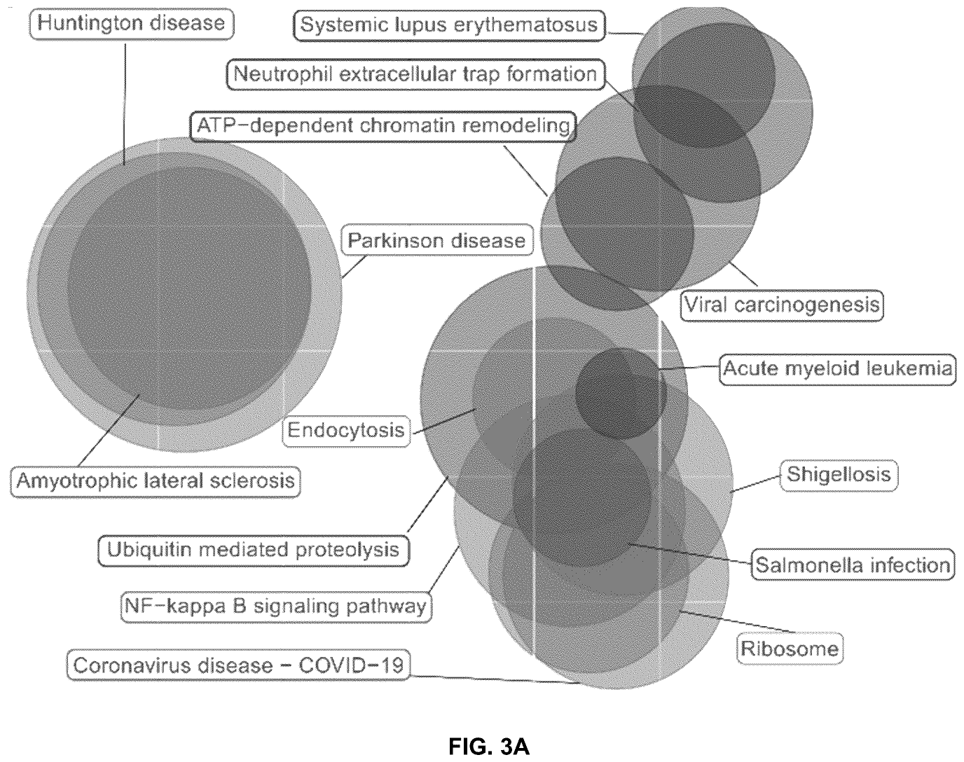

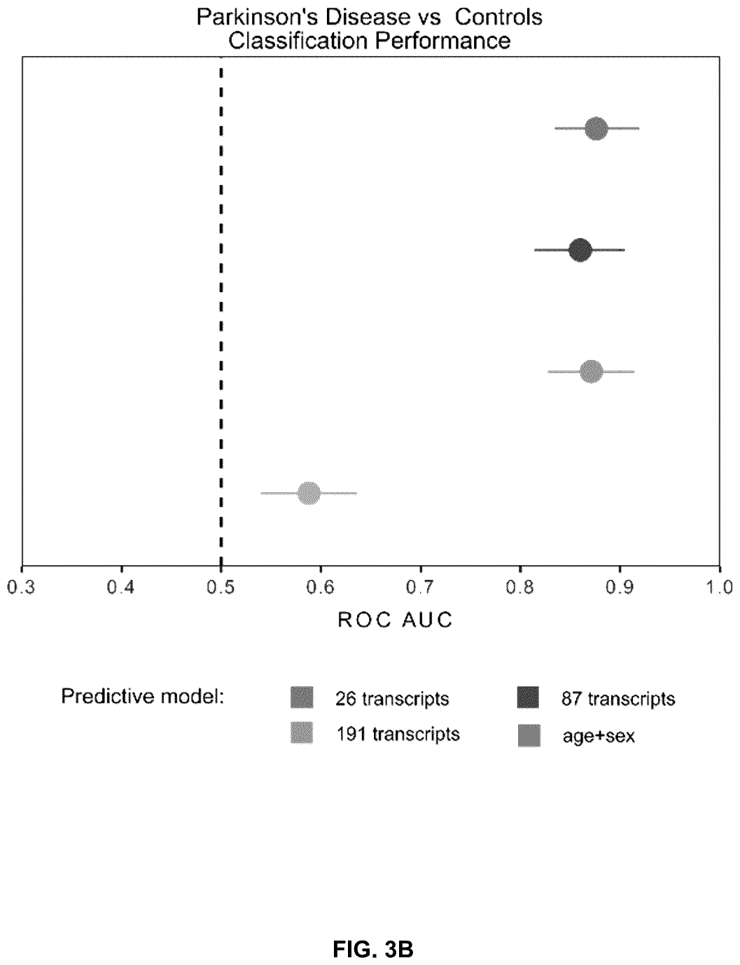

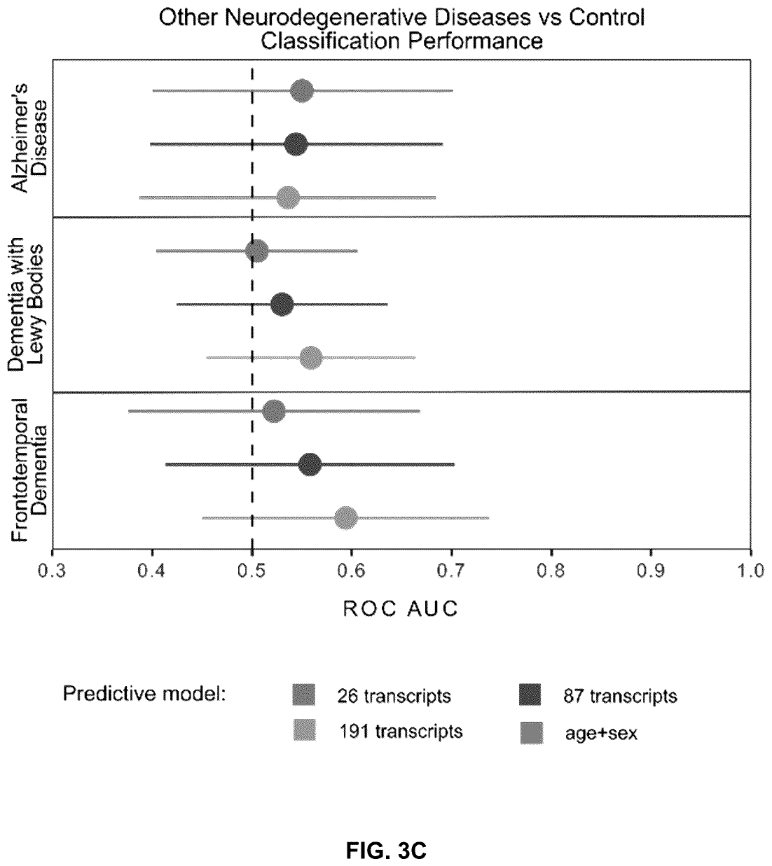

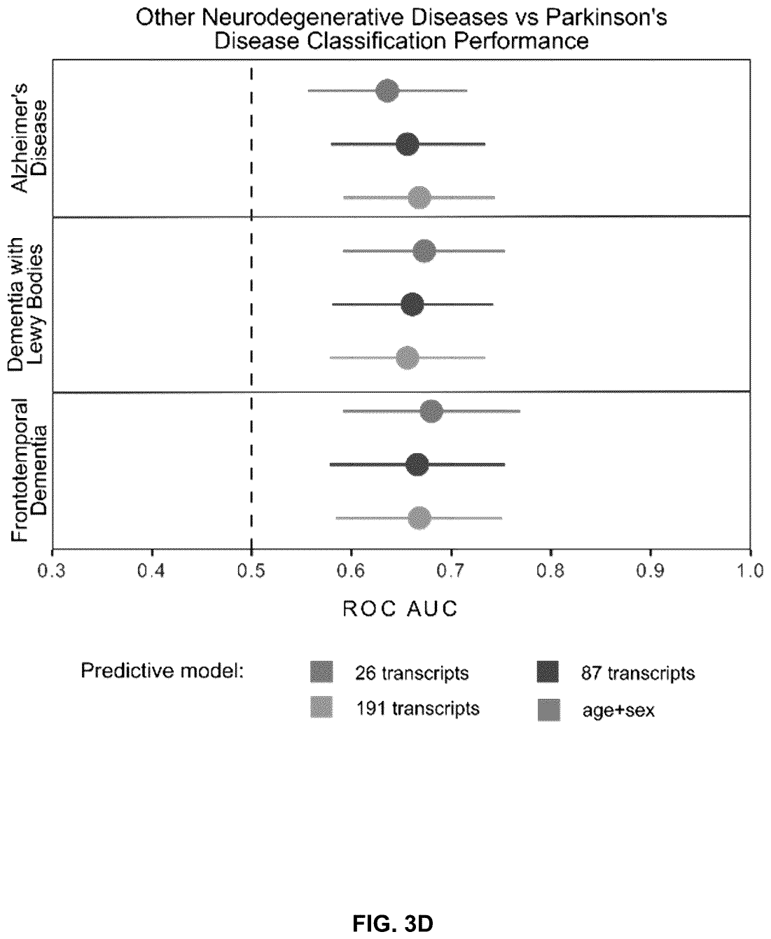

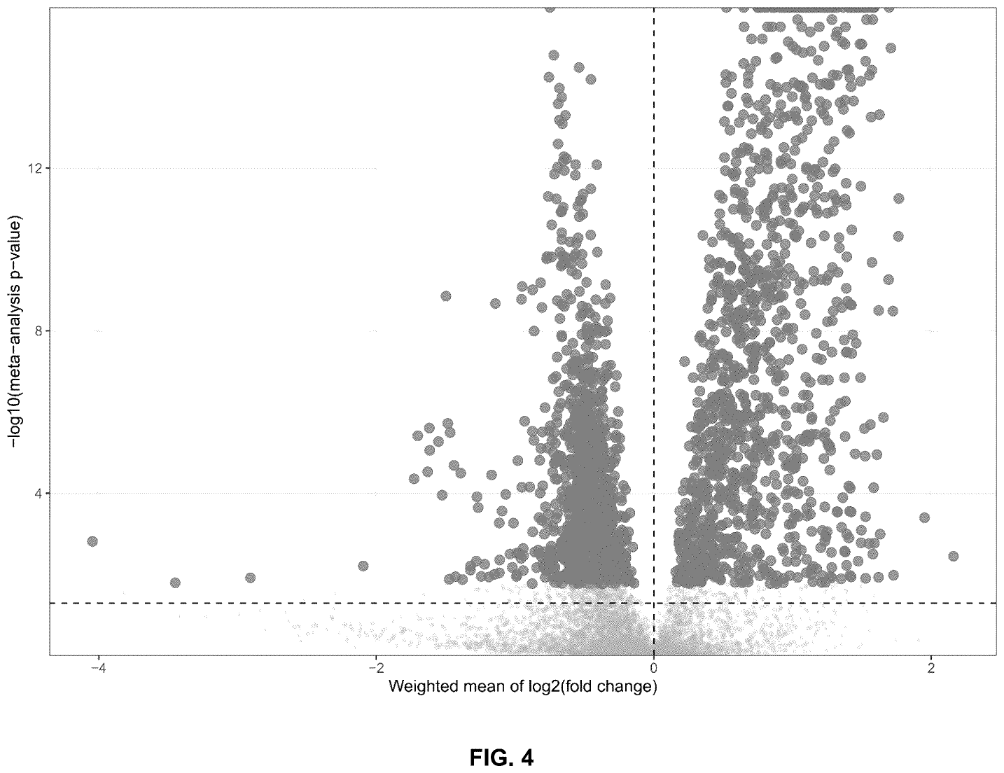

Two independent movement disorder clinics were leveraged (Hospital Universitari Mutua Terrassa (HUMT) and Washington University School of Medicine (WashU)) with a total of 181 PD participants (nHUMT=87; nWashU=94) and 200 healthy control participants (nHUMT=119; nWashU=81) (FIG. 1, Table 2). All PD participants had a clinical diagnosis of PD at the time of sample collection. After stringent quality control (QC), differential expression (DE) analyses was performed comparing PD to healthy control participants using DESeq2, followed by integration of HUMT and WashU results through meta-analysis. Meta-analyses was performed on 6,496 transcripts which had same direction of effect in both HUMT and WashU populations, and identified 2,188 DE transcripts, 1,101 of which were upregulated and 1,087 downregulated (FIG. 4). To evaluate the biological relevance of the DE transcripts, Kyoto Encyclopedia of Genes and Genomes (KEGG) pathway enrichment analyses were performed. Significant enrichment was found in PD (p=9.26×10⁻⁴), endocytosis (p=4.21×10⁻⁶) and ubiquitin mediated proteolysis (p=7.41×10⁻⁵), along with other nervous system diseases such as Huntington's disease (HD; p=6.35×10⁻⁴) and amyotrophic lateral sclerosis (ALS; p=6.10×10⁻⁴; FIG. 3A). Additionally, Gene Ontology (GO) enrichment analyses was performed and enrichment was found in the cellular components primary lysosome (p=4.19×10⁻³) and ubiquitin ligase complex (p=2.12×10⁻³), and biological processes such as late endosome to lysosome transport (p=8.18×10⁻⁴) and exocytosis (p=9.48×10⁻⁶). To further replicate results, publicly available whole blood RNAseq data was used from the Parkinson's Progression Markers Initiative (PPMI) and Parkinson's Disease Biomarkers Program (PDBP) and found 424 of the 2,188 DE transcripts identified in plasma were also DE, and had the same direction of effect, in whole blood (FIG. 2A), which represents a significant overlap (p=4.65×10⁻⁶⁰). These 424 transcripts were enriched in ubiquitin mediated proteolysis (p=8.96×10⁻⁵). Additionally, it was investigated if the abundances of the proteins encoded by the identified RNAs were also significantly different. Plasma proteomic data was leveraged from PPMI generated with Olink. A significant overlap was found (p=5.00×10⁻³) of twelve differentially accumulated proteins out of the 2,188 transcripts DE in plasma (FIG. 2A). Further, six of the twelve proteins (APEX1, CD22, COL6A3, PARP1, SERPINB8 and SKAP1) have the same direction of effect as the respective mRNAs from plasma. Looking back to the pathway analysis, it was found that the twelve proteins were present in the immune response pathways. Two of the twelve acellular transcripts that are differentially accumulated in plasma, at both transcript and protein level, are also DE in whole blood (FIG. 2A).

TABLE 2
Summary demographics of the two populations included in the main analyses.

| | Hospital Universitari Mutua Terrassa | | Washington University School of | |
|---|---|---|---|---|
| | (Barcelona, Spain) | | Medicine (Saint Louis, US) | |
| | | Parkinson's | | Parkinson's |
| | Healthy Controls | Disease | Healthy Controls | Disease |
| Participants (N) | 119 | 87 | 81 | 94 |
| Median age (IQR) | 67.00 (61.00- | 69.00 (65.00- | 74.00 (69.00- | 72.00 (67.00- |
| | 74.00) | 74.00) | 78.00) | 77.00) |
| Female (N, %) | 63 (52.94%) | 35 (43.21%) | 49 (60.49%) | 36 (38.29%) |
| Median UPDRS-III | — | 18.00 (14.00- | — | 24.75 (20.69- |
| (IQR) | | 22.00) | | 30.00) |
| Median MoCA (IQR) | — | — | — | 26.50 (24.00- |
| | | | | 28.00) |

N = Sample size;
IQR = Interquartile Range;
UPDRS = Unified Parkinson's Disease Rating Scale;
MoCA = Montreal Cognitive Assessment;
US = United States

### Expression Patterns in Plasma Follow Those Observed in Brain.

Findings were compared to an in-house brain RNAseq dataset to test whether the changes captured in plasma have their origin in pathological changes taking place in the brain. Out of the 2,188 transcripts, 537 transcripts were found that DE in both plasma and brain (p=4.22×10⁻¹⁰⁵; FIG. 2B), 278 of which were upregulated and 259 downregulated. Of the 537 transcripts, 286 (142 upregulated and 144 downregulated) have the same direction of effect in plasma and brain. Transcripts that were DE in both plasma and brain were enriched in neurodegenerative diseases including PD (p=7.86×10⁻⁷), HD (p=6.27×10⁻⁸), and AD (p=2.16×10⁻⁶), as well as endocytosis (p=8.91×10⁻⁴) and ubiquitin mediated proteolysis (p=2.47×10⁻³).

DE transcripts were next compared with differentially expressed genes derived from three independent single-nucleus RNA-seq datasets from individuals with PD and controls: i) subcortical putamen tissue, ii) anterior cingulate cortex, and iii) in-house and publicly available substantia nigra. Out of the 2,188 transcripts DE in plasma, 222 were also DE in at least one cell type in the subcortical putamen (FIG. 2C). The greatest overlap between plasma and subcortical putamen was in transcripts expressed by ependymal cells (a type of glial cells), with 162 shared DE transcripts, followed by 41 transcripts in GRIK3-enriched neuronal cells and eleven transcripts in astrocytes. Transcripts DE in both plasma and subcortical putamen were nominally enriched in HD (p=3.51×10⁻³) and general pathways of neurodegeneration (p=0.03). Next, 77 DE transcripts were found to be shared between plasma and the anterior cingulate cortex. The greatest overlap between plasma and anterior cingulate cortex was in neuronal cells (47 transcripts), followed by myeloid cells (27 transcripts), while only three transcripts were shared between plasma cfRNA and cortical astrocytes. These transcripts were enriched in PD (p=7.59×10⁻⁴), as well as pathways of neurodegeneration (p=1.35×10⁻³). Of the 222 transcripts shared between plasma and subcortical putamen and 77 transcripts shared between plasma and anterior cingulate cortex, 35 were DE in both subcortical putamen and anterior cingulate cortex (FIG. 2C).

In-house substantia nigra showed 411 DE transcripts in common with plasma (FIG. 2C) when comparing PD cases to healthy controls. These transcripts were enriched in PD (p=3.35×10⁻⁴), endocytosis (p=4.26×10⁻⁵) and ubiquitin mediated proteolysis (p=6.92×10⁻⁴). Similar to subcortical putamen, ependymal cells from the in-house substantia nigra dataset showed the greatest overlap with plasma cfRNA transcripts (245 transcripts). In parallel, 370 of the 2,188 DE cfRNA were DE in the public substantia nigra dataset (FIG. 2C). They were enriched in ubiquitin mediated proteolysis (p=6.06×10⁻⁵), dopaminergic synapse (p=8.19×10⁻³) and general pathways of neurodegeneration (p=9.10×10⁻³). Furthermore, 229 plasma DE transcripts were shared with both the in-house and the public substantia nigra. Additionally, 72 of those 229 were in the same direction of effect in the two substantia nigra datasets.

Finally, using publicly available CSF proteomic data generated with Somalogic (PPMI), it was tested whether any of the DE transcripts correspond to differentially abundant CSF proteins. A significant overlap was uncovered (p=4.90×10⁻⁶) of 89 acellular plasma transcripts and their respective protein products in CSF (FIG. 2B), which were enriched in endocytosis (p=8.10×10⁻⁷) and nominally enriched in HD (p=1.83×10⁻²), and general pathways of neurodegeneration (p=4.40×10⁻²). Of the 89 transcripts whose protein products were differentially accumulated in CSF, 24 (such as ARF3, FURIN, DCTN2, HERC1 and SYNE2) are also DE in bulk brain RNAseq.

Plasma Differentially Expressed Transcripts Originate from PD-Risk Loci.

It was then investigated if any of the transcripts DE in plasma were encoded in a known PD risk loci. A significant overlap was detected of 190 transcripts (p=2.88×10⁻²⁷²) that mapped to PD-associated GWAS loci corresponding to 69 non-overlapping genomic regions (FIG. 2D). An average of 3 (+2) overlapping transcripts were found per genomic region, 27 regions overlapping with only one of the identified transcripts, and 14 regions overlapping five to nine transcripts each. Further, it was found that six DE transcripts overlapped (p=1.47×10⁻⁸) PD-associated loci on the X chromosome. Next, the nominated genes in the PD GWAS locus browser were compared with the DE transcripts identified in the analyses to assess if there is an agreement with the nominated gene. Varying levels of support were found for 121 of the 190 autosomal genes, with an average score of 4.42±1.88. Genes located on the X chromosome cannot be evaluated due to the unavailability of sex chromosome data in the GWAS browser. It was checked if any of the 121 genes were DE in whole blood or brain RNAseq, or if their protein products were differentially accumulated in plasma or CSF. 26 of the 121 genes DE were found in whole blood and 24 of the 121 DE were found in the brain. Similarly, it was observed that corresponding protein products of two of the 121 genes (ITGAM and PARP1) were differentially accumulated in plasma, and four (GCH1, HEXIM2, LGALS3 and UFC1) differentially accumulated in CSF.

cfRNA Captures Transcriptomic Signatures Corresponding to Parkinson's Disease.

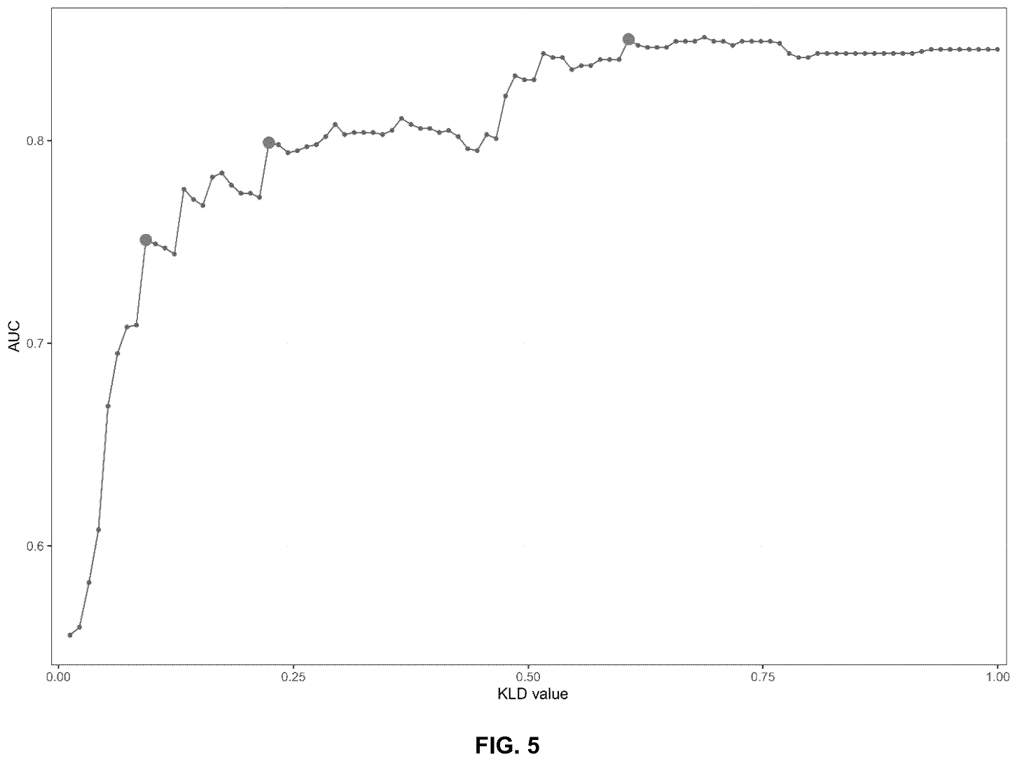

To assess if cfRNA changes can be leveraged to build predictive models, the two independent RNAseq data sets were utilized for the development of predictive models, and an approach was employed similar to that used previously focusing only on the 2,188 DE transcripts. HUMT was used as training and WashU as testing. A total of 100 predictive models were generated corresponding to 100 KLD threshold values (increments of 0.01). Based on the balance between ROC-AUC and number of transcripts included in the model three transcript subsets were selected for further follow up (FIG. 5). Of note, each larger subset is inclusive of the smaller ones. The three selected subsets contained 26, 87 and 191 transcripts (Table 3, Table 4, Table 5), with ROC-AUC values of 0.86, 0.87 and 0.88 in the testing data set (FIG. 3B, Table 6), respectively.

To contextualize the role of transcripts included in the predictive models, it was checked whether any of the transcripts were enriched in the pathways identified in KEGG pathway enrichment analyses described including all the DE transcripts. The smallest subset, consisting of 26 transcripts, captured transcripts that pertain to PD, ubiquitin mediated proteolysis, HD, and ALS (FIG. 3A). The next subset, with 87 transcripts, further captures transcripts involved in endocytosis (FIG. 3A). Next, it was checked whether proteins translated from any of the transcripts included in the predictive models were differentially accumulated in plasma and found two proteins, BMP6 and PARP1, to be differentially accumulated.

Finally, it was explored whether the selected transcripts reflected motor symptom severity, measured by Unified Parkinson's Disease Rating Scale (UPDRS) Part III (UPDRS-III), and cognitive status measured by The Montreal Cognitive Assessment (MoCA). UPDRS-Ill scores were available for both datasets, but not for all participants. Due to sample size (NHUMT=78, nwashU=28), HUMT and WashU data was combined for UPDRS-III analysis and observed that nine (FGR, p=0.04; SH3BP2, p=0.03; ATP5F1B, p=0.04; PTK2B, p-0.02; EMC3, p=0.05; PLAC8, p=8.21×10⁻⁵; TAF10, p=0.04; H2AC11, p=0.01 and H2BC7, p=0.04) of the 191 selected transcripts correlated negatively with UPDRS-III. MoCA information was available for a subset of the WashU population (n=28). Two cfRNA transcripts were found (FCGR3A, p=0.01; RERE, p=0.04) with significant positive correlations to MoCA scores.

TABLE 3
Genes included in predictive model
subsets comprising 26, 87, and 191 transcripts.
Gene Name

| | | | |
|---|---|---|---|
| PSMD4 | ARHGAP27 | RPL37A | PBX1 |
| ARL4C | RPS11 | RPS6 | PRKX |
| GOLGA3 | PLPBP | RBM3 | RPL41 |
| USP10 | UBA52 | RPS6KA1 | RPSA |
| TUBA1A | TUT7 | CALD1 | TRIM33 |
| HCAK1 | RPL13A | DTX3L | |
| TMEM140 | CA198 | EIF4HP1 | |

TABLE 4
Genes included in predictive model
subsets comprising 87 and 191 transcripts.
Gene Name

| | | | |
|---|---|---|---|
| ARHGEF3 | ARF4 | PDK3 | SNRPB |
| BCL2 | IER2 | PSME3 | SNX20 |
| BIRC3 | IL32 | PYGL | SYNE1 |
| BMP6 | JSRP1 | RASAL3 | SYNE2 |
| BTG2 | KANSL1 | RCSD1 | TAF10 |
| CARD8 | MAP3K3 | RPL13P12 | TRIM44 |
| CDC42SE1 | MBD2 | RPL31 | TTC7B |
| CDKN1C | MBOAT2 | RPL34 | TULP4 |
| CDYL | MORC3 | RPS12 | UBA2 |
| CNDP2 | MTMR10 | RPS27P8 | UBTF |
| DHX15 | NACC2 | RPS3AP6 | XRCC5 |
| DOCK8 | NCF2 | RUNX1 | ZBTB7A |
| ELF4 | NCOA2 | SEC31A | ZMAT2 |
| EMC3 | NDUFA4 | SEPTIN5 | SND1 |
| FCGR3A | NPM1P27 | SIPA1L3 | PARP14 |
| FGR | | | |

TABLE 5
Genes included in the predictive model
subset comprising 191 transcripts.
Gene Name

| | | | |
|---|---|---|---|
| ANK1 | FAM110A | MAP3K1 | RPH3A |
| ANKRD9 | FAUP1 | ZFP36L1 | RPL36A |
| ANP32B | FOXN3 | ZNF217 | SBF1 |
| ANXA5 | GDF7 | MCTP1 | SCP2 |
| APP | GGA2 | MPIG6B | SH3BP2 |
| ARL8B | GLUD1 | MT-ND2 | SIAH2 |
| ATM | GPRASP1 | MTR | SLC25A3 |
| ATP5F1B | GTPBP2 | MXD1 | SNRNP200 |
| C10orf95-AS1 | H2AC11 | NCKAP1L | SNRPN |
| CCND2 | H2BC11 | NECAP2 | SPIB |
| CLNS1A | H2BC7 | NUTF2 | SRSF1 |
| CLTC | H2BC9 | PABPC4 | STT3B |
| CORO2B | H4C3 | PARP1 | TAX1BP3 |
| CPB2-AS1 | HSPB1 | PIK3AP1 | THRAP3 |
| CPEB4 | IKZF3 | PLAC8 | TMBIM1 |
| CREBBP | INPP5D | PLCG2 | TOMM20 |
| CTBP1 | IQGAP1 | PNP | TPST2 |
| CTCF | ITCH | PRKD3 | TRIM58 |
| DAD1 | ITGB1P1 | PRRC2A | TSPYL1 |
| DNAJC27 | LBH | PTK2B | TUBA1B-AS1 |
| DNAJC5 | LGALS1 | QKI | UBE2G1 |
| DOCK2 | LGALS8 | RAPGEF1 | VPS37B |
| DUSP6 | LINC01934 | RASSF5 | VSIR |
| EFCAB6 | LST1 | RBM8A | WARS1 |
| EIF3H | MAFB | RERE | YWHAB |
| EIF3L | MAN1A2 | RHBDD1 | ZC3H11A |

TABLE 6
Performance of the three predictive models in PD samples for
the training and the testing datasets (see also FIG. 3(A-D)).

| | | Best | Cohen's | | | | | |
|---|---|---|---|---|---|---|---|---|
| Model | Status | accuracy | kappa | Sensitivity | Specificity | AUC | PPV | NPV |
| 26 transcript | Training | 0.683 | 0.357 | 0.664 | 0.701 | 0.751 | 0.752 | 0.604 |
| model | | | | | | (0.695- | | |
| | | | | | | 0.807) | | |
| | Testing | 0.784 | 0.559 | 0.877 | 0.691 | 0.86 | 0.71 | 0.867 |
| | | | | | | (0.814- | | |
| | | | | | | 0.905) | | |
| 87 transcript | Training | 0.682 | 0.36 | 0.697 | 0.667 | 0.799 | 0.741 | 0.617 |
| model | | | | | | (0.748- | | |
| | | | | | | 0.849) | | |
| | Testing | 0.769 | 0.527 | 0.889 | 0.649 | 0.871 | 0.686 | 0.871 |
| | | | | | | (0.827- | | |
| | | | | | | 0.914) | | |
| 191 | Training | 0.772 | 0.537 | 0.773 | 0.77 | 0.85 | 0.821 | 0.713 |
| transcript | | | | | | (0.805- | | |
| model | | | | | | 0.894) | | |
| | Testing | 0.768 | 0.526 | 0.877 | 0.66 | 0.878 | 0.689 | 0.861 |
| | | | | | | (0.836- | | |
| | | | | | | 0.92) | | |

AUC = area under the curve; PPV = positive predictive value; NPV = negative predictive value.

### The Predictive Models are Specific to PD

To assess whether the predictive models were specific to PD, the models were tested in samples from Dementia with Lewy bodies (DLB, n=29), Alzheimer's disease (AD, n=44), and Frontotemporal dementia (FTD, n=16), using two approaches. Firstly, the models were evaluated with respect to differentiating between each neurodegenerative disease and healthy controls and secondly, if they could discern between PD and DLB, AD, or FTD. The models exhibited low predictive power to differentiate between healthy controls and AD (0.53<AUC<0.55), DLB (0.51<AUC<0.57), or FTD (0.52<AUC<0.59; FIG. 3C, Table 7), confirming that the models are specific to PD. Models performed slightly better in classifying AD (0.64<AUC<0.67), DLB (0.65<AUC<0.67), or FTD (0.67<AUC<0.68; FIG. 3D, Table 7) when compared to PD. Given the know association between APOE genotype and AD risk, it was tested whether the addition of APOE genotype affects the ability of the predictive models to differentiate between AD and PD. With the inclusion of APOE information, an increase was observed in the power of the predictive models to discern between AD and PD (0.85<AUC<0.88).

TABLE 7
Performance of the three predictive models in differentiating between
AD, DLB or FTD and healthy controls or PD (see also FIG. 3(A-D)).

| | | | Best | Cohen's | | | | | |
|---|---|---|---|---|---|---|---|---|---|
| Reference | Model | Status | accuracy | kappa | Sensitivity | Specificity | AUC | PPV | NPV |
| Healthy | 26 | Alzheimer's | 0.515 | 0.028 | 0.5 | 0.529 | 0.55 | 0.636 | 0.391 |
| control | transcript | disease | | | | | (0.4- | | |
| | model | | | | | | 0.701) | | |
| | | Dementia with | 0.479 | −0.022 | 0.476 | 0.483 | 0.505 | 0.824 | 0.154 |
| | | Lewy bodies | | | | | (0.404- | | |
| | | | | | | | 0.606) | | |
| | | Frontotemporal | 0.5 | 0 | 0.5 | 0.5 | 0.522 | 0.636 | 0.364 |
| | | dementia | | | | | (0.376- | | |
| | | | | | | | 0.668) | | |
| | 87 | Alzheimer's | 0.521 | 0.041 | 0.571 | 0.471 | 0.544 | 0.64 | 0.4 |
| | transcript | disease | | | | | (0.398- | | |
| | model | | | | | | 0.691) | | |
| | | Dementia with | 0.551 | 0.059 | 0.551 | 0.552 | 0.53 | 0.862 | 0.195 |
| | | Lewy bodies | | | | | (0.424- | | |
| | | | | | | | 0.636) | | |
| | | Frontotemporal | 0.504 | 0.009 | 0.571 | 0.438 | 0.558 | 0.64 | 0.368 |
| | | dementia | | | | | (0.413- | | |
| | | | | | | | 0.703) | | |
| | 191 | Alzheimer's | 0.527 | 0.055 | 0.643 | 0.412 | 0.527 | 0.643 | 0.412 |
| | transcript | disease | | | | | (0.378- | | |
| | model | | | | | | 0.677) | | |
| | | Dementia with | 0.568 | 0.082 | 0.585 | 0.552 | 0.566 | 0.869 | 0.208 |
| | | Lewy bodies | | | | | (0.462- | | |
| | | | | | | | 0.671) | | |
| | | Frontotemporal | 0.509 | 0.018 | 0.643 | 0.375 | 0.594 | 0.643 | 0.375 |
| | | dementia | | | | | (0.451- | | |
| | | | | | | | 0.736) | | |
| Parkinson's | 26 | Alzheimer's | 0.562 | 0.12 | 0.432 | 0.691 | 0.636 | 0.396 | 0.722 |
| disease | transcript | disease | | | | | (0.556- | | |
| | model | | | | | | 0.716) | | |
| | | Dementia with | 0.604 | 0.177 | 0.517 | 0.691 | 0.673 | 0.341 | 0.823 |
| | | Lewy bodies | | | | | (0.591- | | |
| | | | | | | | 0.754) | | |
| | | Frontotemporal | 0.596 | 0.124 | 0.5 | 0.691 | 0.68 | 0.216 | 0.89 |
| | | dementia | | | | | (0.591- | | |
| | | | | | | | 0.769) | | |
| | 87 | Alzheimer's | 0.563 | 0.119 | 0.477 | 0.649 | 0.656 | 0.389 | 0.726 |
| | transcript | disease | | | | | (0.579- | | |
| | model | | | | | | 0.734) | | |
| | | Dementia with | 0.549 | 0.081 | 0.448 | 0.649 | 0.661 | 0.283 | 0.792 |
| | | Lewy bodies | | | | | 0.581- | | |
| | | | | | | | 0.742) | | |
| | | Frontotemporal | 0.606 | 0.126 | 0.562 | 0.649 | 0.666 | 0.214 | 0.897 |
| | | dementia | | | | | (0.578- | | |
| | | | | | | | 0.754) | | |
| | 191 | Alzheimer's | 0.637 | 0.25 | 0.614 | 0.66 | 0.669 | 0.458 | 0.785 |
| | transcript | disease | | | | | (0.593- | | |
| | model | | | | | | 0.745) | | |
| | | Dementia with | 0.554 | 0.091 | 0.448 | 0.66 | 0.653 | 0.289 | 0.795 |
| | | Lewy bodies | | | | | (0.576- | | |
| | | | | | | | 0.731) | | |
| | | Frontotemporal | 0.642 | 0.17 | 0.625 | 0.66 | 0.665 | 0.238 | 0.912 |
| | | dementia | | | | | (0.581- | | |
| | | | | | | | 0.749) | | |

AUC = area under the curve; PPV = positive predictive value; NPV = negative predictive value.

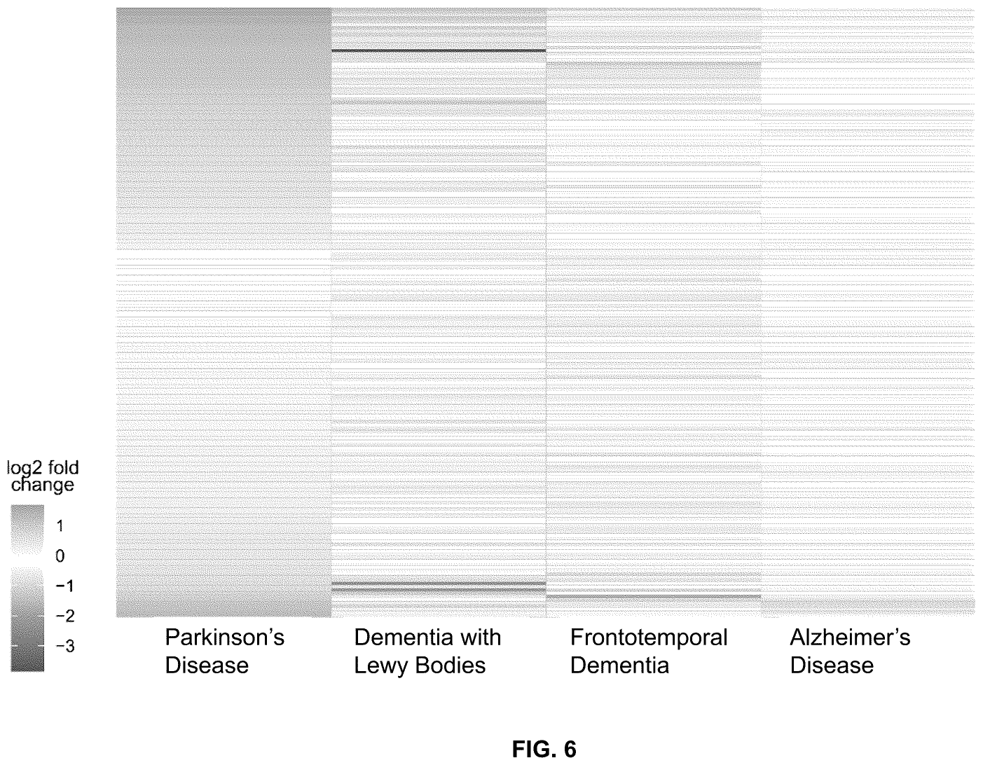

To gain more insight about whether any of the transcripts included in the predictive models were commonly dysregulated across different NDDs, plasma expression patterns were investigated across PD, DLB, AD and FTD. The most remarkable difference was observed in FTD, where the same transcripts seem to be dysregulated in opposite direction to PD (FIG. 6).

### DISCUSSION

According to the present disclosure, two independent datasets were leveraged to identify plasma cfRNAs that were dysregulated in PD participants and, for the first time in PD, employ plasma cfRNAs to develop predictive models that can distinguish between PD and healthy controls. Overall, 2,188 transcripts that were DE were identified in plasma of PD participants. Through pathway analyses the identified transcripts were shown to be part of PD-associated pathways, such as endocytosis, ubiquitin mediated proteolysis, PD, and PD-related cellular components and biological processes such as ubiquitin ligase complex, primary lysosome, lysosome transport and exocytosis. Furthermore, the findings in plasma are consistent with those in blood by utilizing publicly available PD blood bulk RNAseq data to replicate 424 of the findings. Additionally, protein products of twelve of the identified acellular transcripts are also dysregulated in plasma. One of the twelve transcripts, COL6A3, has previously been indicated in other neurologic disorders, namely muscular dystrophy and dystonia. Interestingly, while they have not been directly implicated in PD previously, three of the twelve transcripts, ITGAM, SERPINB8, and SLC27A4, are associated with dry, flaky skin, which is a common symptom of PD, further supporting the biological relevance of the findings of the present disclosure.

Leveraging an in-house brain bulk RNAseq dataset, it was shown that plasma cfRNA captures changes occurring in the brains of PD participants, most likely due to blood-brain barrier (BBB) leakage. Moreover, a significant overlap was found between transcripts DE in plasma and the corresponding proteins in CSF. Specifically, 24 differentially accumulated CSF proteins are produced from transcripts DE in both plasma and brain. Several of these proteins/transcripts are associated with other neurodegenerative or movement disorders, such as AD (KLC1) and dystonia (SYNE2), or PD-related pathologies and pathways (ARF3, DCTN2, HERC1). Specifically, ARF3 contributes to the disruption of Golgi apparatus and there is evidence of Golgi fragmentation in PD. DCTN2 colocalizes with phosphorylated SNCA in Lewy bodies in participants with PD and DLB. HERC1 is an E3 ubiquitin protein ligase whose dysregulation leads to alterations in the endosomal system, and both atypical ubiquitination and dysfunction of endosomal system are hallmarks of PD pathobiology. In consequence, it is plausible to think that some of the identified transcripts in plasma might have origin in the central nervous system.

Lastly, 196 of the 2188 identified transcripts were shown as originating from genomic regions associated with PD. Of those, 121 have some level of support as the driving gene for a given locus. Remarkably, six of these transcripts are encoded by genes nominated by the PD GWAS studies (ADORA2B, DYRK1A, GCH1, PNA1, MCCC1, TMEM163), adding additional evidence to the already prioritized genes. All but ADORA2B had high supporting scores, ranging from seven (MCCC1) to ten (DYRK1A), in the GWAS browser. Interestingly, DYRK1A has the same level of support as SNCA, a well-known PD-associated gene, in the locus browser, adding evidence of the involvement of DYRK1A in the pathobiology of PD and the pathological relevance of improved transcriptomic plasma biomarkers for PD.

To date, there are no established biomarkers for accurate PD diagnosis, yet with α-synuclein seed amplification assays (SAA) having promising performance in CSF. The identified DE transcripts were utilized to develop minimally-invasive predictive models capable of differentiating between PD and healthy controls. Three scalable models were developed with high predictive power to classify PD, with AUC values between 0.86 and 0.88. Two of the predictive transcripts, BMP6 and PARP1, are also dysregulated on a protein level in plasma, which opens additional possibilities to leverage the proteins as biomarkers. Interestingly, increased levels of BMP6 have been associated with AD, while PARP1 is connected with α-synuclein pathology and PD, but they have not been explored as biomarkers. Regardless, the present disclosure demonstrates the feasibility of developing transcriptomic models are specific to PD, as they are unable to differentiate between DLB, FTD or AD and healthy controls and capture pathways relevant to the known pathobiology of PD. Notably, the predictive transcripts are dysregulated in FTD in the opposite direction to PD, suggesting that underlying molecular pathways could be shared between PD and FTD, though regulated differently, similar to what has been observed previously for AD and DLB. Due to the complexity of PD, and the biological interplay between DNA, RNA, and protein functions, additional PD biomarkers can benefit from integrating proteomic and transcriptomic data. Mapping the predictive model transcripts to the pathway analysis results, model transcripts are involved in PD relevant pathways, such as PD, ubiquitin mediated proteolysis, as well as other nervous system disorders, like HD and ALS, adding further evidence of the biological relevance of these models and the pathological overlap across neurodegenerative diseases.

Nine transcripts, FGR, SH3BP2, ATP5F1B, PTK2B, EMC3, PLAC8, TAF10, H2AC11, and H2BC7, included in the models significantly negatively correlate with motor symptoms measured by UPDRS-III scale, several of which have known links to PD. PTK2B is highly expressed in the nervous system and has been indicated in AD for its role in synaptic homeostasis. In PD, PTK2B is associated with variant rs11060180 (p=1.12×10⁻⁴), considered to be a PD-risk allele. Similarly, ATP5F1B has no known correlation to PD, but is associated with dystonia, which, like PD, is a movement disorder, further supporting its potential relation with UPDRS-III. Finally, two transcripts, RERE and FCGR3A, correlate with dementia symptoms measured by MOCA scores. The latter of the two have already been associated with memory disorders, supporting biological relevance of the predictive models. Together, this further shows that acellular transcripts are truly capturing PD pathology and the complexity of the movement disorder diseases and potentially points to different mechanisms causing the motor versus cognitive symptoms in PD participants.

This study shows that cfRNAs have a potential to aid in diagnosis of PD as cost effective, minimally invasive biomarkers. Several plasma transcripts were identified that have already been associated to PD or relevant pathways, correlate with symptom severity, and could potentially be leveraged for disease monitoring. On top of that not only are some of those transcripts dysregulated in different tissues, but they are also encoded in known PD loci. Additionally, some of them result in alterations in protein abundance when compared to healthy controls in relevant tissues. Overall, the present disclosure has demonstrated that cfRNA has the power to capture changes relevant to PD pathology, enables translation to the clinic, and when replicated and validated in larger samples sizes, benefits the whole PD community by providing non-invasive biomarkers that are cost-effective and can be implemented in remote areas, providing access to care for all.

What is claimed is:

1. A method for treating Parkinson's Disease in a subject in need thereof, the method comprising:
providing a first biological sample obtained from the subject;
measuring a first level of at least one plasma cell-free RNA (cfRNA) transcript in the first biological sample; and
determining a first treatment for the subject based on the level of the at least one plasma cfRNA transcript.

2. The method of claim 1 further comprising determining a PD severity level based on the level of the at least one plasma cfRNA transcript.

3. The method of claim 2, wherein determining the treatment for the subject is further based further on the determined PD severity level.

4. The method of claim 1 further comprising determining a continuing treatment for the subject, wherein determining the continuing treatment comprises:
providing a second biological sample obtained from the subject, wherein the second biological sample is obtained from the subject at a later time than the first biological sample is obtained from the subject;
measuring a second level of the at least one plasma cfRNA transcript in the second biological sample; and
determining a continuing treatment for the subject based on a change between the first and second levels of the at least one plasma cfRNA transcript.

5. The method of claim 1, wherein the at least one cfRNA transcript is a combination of at least 26 cfRNA transcripts.

6. The method of claim 5, wherein the combination of at least 26 cfRNA transcripts comprises FGR, SH3BP2, ATP5F1B, PTK2B, EMC3, PLAC8, TAF10, H2AC11, H2BC7, RERE, FCGR3A, and APOE.

7. The method of claim 6, wherein the at least one cfRNA transcript is a combination of at least 87 cfRNA transcripts.

8. The method of claim 7, wherein the at least one cfRNA transcript is a combination of at least 191 cfRNA transcripts.

9. A method of differentiating Parkinson's Disease (PD) from a non-PD neurodegenerative disease in a subject in need thereof, the method comprising:
providing a biological sample from the subject;
measuring a level of at least one plasma cell-free RNA (cfRNA) transcript in the biological sample; and
determining whether the subject has PD or a non-PD neurodegenerative disease based on the level of the at least one plasma cfRNA transcript.

10. The method of claim 9, wherein the subject is selected from a subject having, suspected of having, or at risk for developing PD and a subject having, suspected of having, or at risk for developing a non-PD neurodegenerative disease.

11. The method of claim 10, wherein the non-PD neurodegenerative disease comprises Alzheimer's disease (AD), dementia with Lewy bodies (DLB), frontotemporal dementia (FTD), multiple system atrophy (MSA), progressive supranuclear palsy (PSP), Huntington's Disease (HD), and amyotrophic lateral sclerosis (ALS).

12. The method of claim 9 further comprising, when the subject is determined to have PD, determining a PD severity level based on the level of the at least one plasma cfRNA transcript.

13. The method of claim 12 further comprising determining a treatment for the subject based further on the determined PD severity level.

14. The method of claim 9, wherein the at least one cfRNA transcript is a combination of at least 26 cfRNA transcripts.

15. The method of claim 14, wherein the combination of at least 26 cfRNA transcripts comprises FGR, SH3BP2, ATP5F1B, PTK2B, EMC3, PLAC8, TAF10, H2AC11, H2BC7, RERE, FCGR3A, and APOE.

16. The method of claim 15, wherein the at least one cfRNA transcript is a combination of at least 87 cfRNA transcripts.

17. The method of claim 16, wherein the at least one cfRNA transcript is a combination of at least 191 cfRNA transcripts.

18. A predictive model to identify Parkinson's Disease (PD) in a subject in need thereof, wherein the predictive model comprises:
providing a level of at least one plasma cell-free RNA (cfRNA) transcript measured from a biological sample obtained from the subject;
calculating a Kullback-Leibler divergence (KLD) value for each cfRNA transcript;
ranking the KLD value for each cfRNA transcript;
generating a L2 regularization linear model based on the ranked KLD values;
computing an area under a curve (AUC) value for the L2 regularization linear model, wherein the curve is a receiver operating characteristic (ROC) curve;
comparing the computed AUC value to a threshold value; and
identifying PD in the subject if the AUC value meets the threshold value.

19. The predictive model of claim 18 further comprising, when PD is identified in the subject, determining a treatment for the subject based on the computed AUC value.

20. The predictive model of claim 18, wherein the threshold value is 0.85.
